(19) United States
(12) Patent Application Publication
(10) Pub. No.: US 2008/0188923 A1
(43) Pub. Date: Aug. 7, 2008

(54) ENDOVASCULAR DEVICES TO PROTECT ANEURYSMAL WALL

(76) Inventor: Jack Fa-De Chu, Santa Rosa, CA (US)

Correspondence Address: JACK CHU, 5918 SUNHAWK DRIVE, SANTA ROSA, CA, 95409, US

(21) Appl. No.: 12/021,249

(22) Filed: Jan. 28, 2008

Related U.S. Application Data
(60) Provisional application No. 60/887,723, filed on Feb. 1, 2007.
(60) Provisional application No. 60/889,564, filed on Feb. 13, 2007.

(51) Int. Cl. A61F 2/82

(52) U.S. Cl. 623/1.15

(57) ABSTRACT

Methods and systems for preventing aneurysm rupture and reducing the risk of migration and endoleak are disclosed. Specifically, an inflatable multiple walls liner is applied directly to treat the interior of the aneurysm site. Also disclosed are methods to deliver the inflatable multiple walls liner directly to treatment sites.

This application claims the benefit of U.S. Provisional Application No. 60/887,723, which was filed Feb. 1, 2007, and U.S. Provisional Application No. 60/889,564, which was filed Feb. 13, 2007, the disclosure of which is incorporated herein by this reference.

### FIELD OF THE INVENTION

Methods and devices for preventing rupture of an aneurysm and reducing the risk of endoleak are disclosed. Specifically, methods and systems for applying inflatable multiple-layer liners directly to treatment sites and to the interior of the vessel wall are provided.

### BACKGROUND OF THE INVENTION

An aneurysm is a localized dilation of a blood vessel wall usually caused by degeneration of the vessel wall. These weakened sections of vessel walls can rupture, causing an estimated 32,000 deaths in the United States each year. Additionally, deaths resulting from aneurysmal rupture are suspected of being underreported because sudden unexplained deaths are often misdiagnosed as heart attacks or strokes while many of them may in fact be due to ruptured aneurysms.

Approximately 50,000 patients with abdominal aortic aneurysms are treated in the U.S. each year, typically by replacing the diseased section of vessel with a tubular polymeric graft in an open surgical procedure. However, this procedure was risky and not suitable for all patients. Patients who were not candidates for this procedure remained untreated and thus at risk for aneurysm rupture or death.

A less-invasive procedure is to place a stent graft at the aneurysm site. Stent grafts are tubular devices with one or more metallic stents attached to the polymeric grafts such as Dacron® or ePTFE film. The metallic stent is generally stitched, glued or molded onto the biocompatible tubular covering and provides strength to the graft. Additional features such as barbs and hooks on the stent can enhance the graft's ability to anchor in the vessel. In other embodiments, one or more inflatable channels were attached to the tubular graft for additional strength, and, in some cases, replaced the metal scaffold. The size of the tubular graft is usually matched to the diameter of the healthy vessel adjacent to the aneurysm. Usually, stent grafts can be positioned and deployed at the site of an aneurysm using minimally invasive procedures. Essentially, a delivery catheter having a tubular graft compressed and packed into the catheter's distal tip is advanced through an artery to the aneurismal site. The tubular graft is then deployed within the vessel lumen in juxtaposition to the diseased vessel wall, and forming a flow conduit without replacing the dilated section of the vessel. This new flow conduit insulates the aneurysm from the body's hemodynamic forces, therefore decreasing hemodynamic pressure on the disease portion of the vessel and reducing the possibility of aneurysm rupture.

While tubular stent grafts represent improvements over more invasive surgery procedures, there are still risks associated with their use to treat aneurysms. Stent graft migration and endoleak are the biggest challenges for tubular stent grafts because of the hemodynamic forces within the stent graft lumen, limited fixation near the neck, and the lack of lateral support for the stent graft at the aneurysm site. Frequently, most of the support for the tubular stent graft depends on its fixation on a very limited section of healthy vessel between the renal artery and the aneurysm, i.e. at the neck of the aneurysm. The aneurysm sac between the aneurysm wall and the tubular stent graft is usually filled with blood or unorganized thrombosis and provides little or no support to the stent graft which is under a constant hemodynamic force. Stent graft migration is especially common in aneurysms when there is insufficient overlap between the stent graft and the vessel and in tortuous portions of the vessels where asymmetrical hemodynamic forces place uneven forces on the stent graft.

Stent graft migration can break the seal between the tubular stent graft and vessel and lead to Type I endoleak, or the leaking of blood into the aneurismal sac between the outer surface of the stent graft and the inner surface of the blood vessel. This endoleak can result in the aneurysm wall being exposed to hemodynamic pressure again, thus increasing the risk of rupture. It would be beneficial to have devices and methods that protect the aneurysm and reduce the risk of post implantation device migration and endoleak.

Other than Type I endoleak, many patients experience some other issues after undergoing stent graft therapy for their aneurysms. Type II endoleak is defined as the leakage due to patent collateral arteries in the aneurismal sac. The patent collateral arteries (inferior mesenteric artery, lumbar artery) in the aneurismal sac can lead to an increased pressure in the aneurysm and may cause aneurysm enlargement and rupture in some patients. Type III and IV endoleaks are leaks caused by defects in the stent grafts. As a result, physicians often have to follow up closely with patients after endovascular therapy and perform secondary intervention to stop the leakage if it is required. Both follow-up procedures and secondary interventions are undesirable because the cost and the risk involved in those procedures.

Based on the foregoing, one goal of treating aneurysms is to provide a therapy that does not migrate or leak. To achieve this goal, stent grafts with anchoring barbs or hooks that engage the vessel wall have been developed to enhance their attachment to the wall as described in U.S. patents and patent applications U.S. Pat. Nos. 6,395,019B2, 7,081,129B2, 7,147,661B2, 2003/0216802A1. Additionally, endostaples that punch through both graft and vessel wall to fix grafts to the vessel wall have been developed. U.S. Pat. No. 6,007,575 and U.S. Patent Application Publication No. 2003/0093145A1 disclose the use of protruded features on the surface of inflated channels to increase the friction and fixation between the graft and the vessel wall. While these physical anchoring devices have proven to be effective in some patients, stent grafts failure and migration are still reported in many patients.

An additional way to reduce the risk of stent graft migration is to add growth factors or fibril to the surface of the stent graft to promote cells or tissue to grow onto the stent graft. The attached cells or tissue on the stent graft can enhance the bonding between the vessel wall and the stent graft and increase its fixation on the vessel wall. However, the amount of tissue growth required to secure the stent graft on the vessel wall is uncertain at this moment.

Other than the improvement of the stent graft, several attempts have been made to prevent endoleak by embolizing the aneurismal sac with thrombosis or fillers such as coils, gel, fibers, etc. U.S. Pat. Nos. 6,658,288 and 6,748,953 discussed the methods to use electrical potential to create thrombosis in the aneurysm. U.S. patents and patent applications U.S. Pat. Nos. 5,785,679, 6,231,562, 6,613,037, 7,033,389, 637,973, 6,656,214, 633,100, 6,569,190, 2003/135264A1, 36745A1, 44358A1, 2005/90804A1 and WO95/08289 disclose methods and devices to embolize the aneurismal sac. Those methods and devices create hardened material in the aneurismal sac to prevent endoleaks. However, embolization agent or dislodged emboli can travel downstream and embolize small vessels in the legs or colon. As a result, a stent graft or a barrier layer is usually utilized to exclude the aneurismal sac from the major blood conduit before injecting embolization agent into the aneurismal sac. This approach reduces the chance for the emboli to pass through the barrier layer and travel to the iliac arteries. However, the junctions to the collateral vessels in the aneurismal sac are not protected. Physicians usually will occlude the patent collateral vessels before the embolization procedure. Unfortunately, it is very difficult to identify the patency of the collateral vessels (inferior mesenteric artery, lumbar artery) in the aneurismal sac by the current imaging techniques, such as CT or MRI. If those collateral vessels are patent, i.e. a Type II endoleak is diagnosed, there is a risk that the injected embolization agent or dislodged emboli will migrate into those collateral vessels and embolize important vessels in the lumbar and colon.

Due to the risk of accidental embolization, some have proposed that the injected filler is contained in a graft or a membrane and the aneurismal sac be isolated before the injection of filler, as disclosed in U.S. patent and patent application Nos. U.S. Pat. Nos. 6,729,356, 5,843,160, 5,665,117, 2004/98096A1 and 2006/212112A1, which are fully incorporated by reference herein. The fill structure generally has a spherical shape, and there is typically a tubular main conduit in the middle for restoring the original geometry of the flow conduit. However, there are several concerns with this approach. First, to avoid endoleaks and migration, a close contact between the outer wall of the fill structure and the aneurysm wall is important to seal the junctions of the aorta to the origins of the collateral branch arteries. Because the fill structure is constrained by the aneurysm wall and the stent graft (or a shaping balloon) in the middle, it is essential to inject sufficient amount of filler in the fill structure to maintain close contact between the aneurysm wall and fill structure and, at the same time, avoid injecting excess amount of filler and exerting additional stress on the weak aneurysm wall. However, the gap between the fill structure and the aneurysm wall cannot be visualized easily (no contrast agent in gap or aneurysm wall) under Fluoroscope during the inflation of the fill structure, physician cannot determine if the gap has been filled (or not being filled) by the fill structure. This uncertainty can cause excess amount of filler in the fill structure and consequently high stress on the aneurysm wall and place the patient in great risk. Additionally, the aneurysm is usually sealed by a stent graft or a lumen shaping balloon before the fill structure is inflated. Existing blood in the aneurysm (with the added filler) can also cause high stress on the aneurysm wall during the inflation of fill structure if the collateral arteries in the aneurysm are occluded. Second, a significant amount of filler is required to fill the aneurismal sac for patients with large aneurysms. The effect of this large chunk of filler on vessel movement and the adjacent organs is still unknown. Third, the aneurysm tends to remodel and possibly to shrink after the placement of filler and/or stent graft as a result of the reduced hemodynamic pressure in the aneurysm. The flow conduit within the fill structure may be compressed by the remodeled aneurysm and become smaller if the fill structure can't resist the compression. This may cause occlusion or a higher hemodynamic pressure on the fill structure and lead to migration from its designated position.

Thus, there is a need to develop a new method to treat an aneurysm site to protect the aneurysm and reduce the risk of endoleak and rupture. The present invention addresses this opportunity by providing methods and systems to protect the aneurysm and to reduce the likelihood of endoleak, migration and rupture at aneurysm sites.

### SUMMARY OF THE INVENTION

The present invention addresses the issues with the current therapies by providing methods and systems to reduce the likelihood of migration, endoleak and rupture at aneurysm sites. The systems comprise an inflatable multiple walls liner which is larger or the same size as the aneurysm. This inflatable multiple walls liner is flexible with an outer wall and an inner wall. After the liner is introduced in the aneurysm, the conformation of the liner to the aneurysm wall is achieved by the flexible walls and a hemodynamic force. During the inflation of the liner, the outer wall of the liner remains in close contact with the aneurysm wall. The inner wall of the liner expands away from the inner surface of the aneurysm in a restrained fashion by the connectors between the walls and defines the flow conduit. Additional filler increases the thickness of the liner without exerting excess circumferential force against aneurysm wall. After the liner is deployed in the aneurysm, the shape of the flow conduit is determined by the shape of the aneurysm, connector and the thickness of the liner.

In one embodiment of the present invention, the inflatable multiple walls liner has two openings. The materials used for the walls are flexible and significantly inelastic so that they can conform to the inner surface of the aneurysm. The space between the outer and inner walls comprises at least one inflatable chamber to be filled by the injected filler. The walls and connectors between the walls define the inflatable chamber and its thickness. The inner wall determines the blood flow conduit with a first opening and a second opening. After deployed in the aneurysm, the blood flow conduit has a shape determined by the inner surface of the aneurysm, connector, and the thickness of the liner. This invention is particularly suitable for treating patients with Thoracic aortic aneurysm (TAA), aneurysms in the peripheral arteries, or abdominal aortic aneurysms (AAA) with some distance from the iliac bifurcation.

In the second embodiment of this invention, the inflatable multiple walls liner is made of flexible pouch shape walls. Each wall can be made from the same or different material. The walls are connected by a stripe, a string or a bond, such as glue bond, weld bond, heat bond, etc. at a plurality of locations between the walls. The material used for the connector should have a significant inelasticity to avoid excess stretching during inflating. The extent of the connection can be a single point, an area, a line, or a dotted line. Combined with the walls, the arrangement and the type of connector define the inflatable chamber and are important for the flexibility of the liner. If the connector is long, the liner is thick with a lower flexibility after inflation. If a glue bond is used as the connector between the inner and outer walls, the connector is short, and the liner is thin with a higher flexibility at the connector. It is preferable that the liner is relatively thinner near the opening of the flow conduit to increase its flexibility to comply with patient's anatomy near the opening for optimum seal. On the other hand, the inflatable multiple walls liner can be thicker in the middle of the aneurysm for additional strength and aneurysm protection.

In another embodiment of this invention, inflatable multiple walls liner can be formed by attaching a plurality of inflatable patches on either surface of a pouch shape wall. Each inflatable patch is an inflatable chamber to be filled by the filler and is in fluid communication with adjacent inflatable chamber. The inflatable patch is not permeable to the injected filler. The attachment of inflatable patch to the wall can be done by sewing, stitching, glue bond, weld bond, heat bond, etc. Alternatively, at least one side of the inflatable patch is bonded to an adjacent inflatable patch.

In another embodiment of this invention, the inflatable multiple walls liner can be formed by bonding a plurality of inflatable channels either to themselves or to a pouch shape wall. Each inflatable channel is an inflatable chamber to be filled by the filler and is in fluid communication with adjacent inflatable chamber. The inflatable channel is not permeable to the injected filler and inflatable by the filler. The bonding of inflatable channels can be done by glue bond, weld bond, heat bond, etc. Alternatively, inflatable channel can be attached to either side of a pouch shape wall to form an inflatable multiple walls liner.

In another embodiment of this present invention, the inflatable multiple walls liner is created by combining inflatable chambers of various forms such as inflatable patch or inflatable channel. The same filler material can be used to inflate inflatable chambers in the liner. Alternatively, inflatable chambers can be filled by different fillers to achieve the optimum performance. For example, inflatable chamber facing the aneurysm wall can be filled with soft filler with a better cushion to the aneurysm wall, and inflatable chamber facing the flow conduit can be filled with hard filler with a better support to the flow conduit.

In another embodiment of the present invention, the inflatable multiple walls liner is particularly suitable for lining aneurysm close to the bifurcation, especially abdominal aortic aneurysms (AAA) adjacent to the iliac bifurcation. The walls of the liner are flexible with three openings. The space between the outer and inner walls defines at least one inflatable chamber to be filled by the filler. One or more connectors between the walls define the thickness of the inflatable chamber and the liner. The inner wall of the liner determines the blood flow conduit with one inlet and two outlets. After deployed in the aneurysm, the liner would have the shape defined by the inner surface of the aneurysm. The blood flow conduit would have a shape determined by the inner surface of the aneurysm, connector and the thickness of the liner.

In another embodiment of the present invention, the inflatable multiple walls liner is particularly suitable for lining aneurysm which has extended from aorta to the iliac artery. The walls of the liner are flexible with a bifurcation and two sleeves. The space between the outer and inner walls defines at least one inflatable chamber to be filled by the injected filler. One or more connectors between the walls define the thickness of the inflatable chamber and the liner. The inner wall defines the blood flow conduit with one inlet and two outlets. After deployed in the aneurysm, the liner would have the shape defined by the inner surface of the aneurysm. The blood flow conduit would have a shape determined by the inner surface of the aneurysm, connector and the thickness of the liner.

In yet another embodiment of the present invention, the systems to treat aneurysm also include at least one stent which is placed near the opening of the liner after the liner is deployed in the aneurysm. Preferably, the stent is deployed at the junction between the liner and the vessel wall to ensure no gap between them. Usually, the stent is most useful to be deployed at the inlet of the blood flow conduit. Optionally, stent can be deployed at the outlet of the blood flow conduit. Alternatively, portion of the stent can be covered with a graft or a membrane to further assist the sealing between the liner and vessel wall. Alternatively, one or more stents can be fixed to the liner by sewing, stitching, glue bond, weld bond, heat bond, etc.

In the practice, physician needs to determine the appropriate liner to use in each patient. Through the imaging techniques such as CT scan or MRI, the size and length of the patient's aneurysm can be measured accurately. Then, the physician can select a liner that best fit the patients' aneurismal anatomy. It is preferred to use a liner with outer diameter no less than the largest inner diameter of the aneurysm. Because the flexible walls of the liner and the hemodynamic force in the liner, the liner will remain conform to the inner surface of the aneurysm.

For a preferred deployment method of this invention, a delivery catheter is used to deliver a multiple walls liner in an aneurysm. The expandable element (e.g. distal balloon) on the delivery catheter is preferable to be of annular shape allowing blood flow through the balloon after inflation. In the collapsed configuration, portion of the liner is placed on top of the distal balloon with its inner wall against the balloon. The end of a feeding tube is inserted in a one way valve within the liner. After the liner and distal balloon are both collapsed into the low profile configurations, they can be compressed and loaded into a sheath on the catheter and sterilized with various known sterilization methods. Then, the liner delivery system can be positioned in the aneurysm site via iliac artery with minimum invasivity. It is preferable that the distal balloon on the distal end of the catheter is deployed near the neck of the aneurysm to ensure that no excess stress is applied on the aneurysm wall. After the distal balloon is deployed, portion of the liner near the inlet is pressed against the vessel wall by the inflated balloon. At the same time, blood flows through the lumen in the distal balloon to expand the liner radially toward the aneurysm wall. As the sheath is retrieved to expose the liner, the expansion continues until the liner covers the whole inner surface of the aneurysm. This procedure is safe because the pressure to expand the liner is the same pressure existed in the aneurysm before the operation. No additional stress is placed on the aneurysm wall during the expansion of the liner. After the inner surface of the aneurysm wall is completely covered by the liner, a second expandable element (e.g. proximal balloon) is inflated at the junction between the liner and the vessel. This proximal balloon can be on the same multi-lumen catheter or on a separate one. The purpose of this proximal balloon is to ensure the patency of blood flow conduit during the inflation of liner. The inflation of the liner gives addition strength to the liner and protects the aneurysm. It is accomplished by injecting fluid filler into the liner through a lumen in the catheter and the feeding tube. As the liner is inflating, the status of inflation is monitored by the radiopaque markers on the liner. Because the outer wall of the liner is already conformed to the inner surface of the aneurysm wall, the injected filler actually moves the inner wall of the liner away from the aneurysm wall. After the appropriate liner thickness is reached, the feeding tube is retrieved from the body, and the filler is encapsulated in the liner. Finally, the balloons are deflated and retrieved from the patient's body with the delivery catheter. Optionally, one or more stents or membrane covered stents are placed at junction between the liner and the vessel wall to ensure seal.

In an alternative deployment method of this invention, a multi-lumen catheter is used to deliver a stent attached liner in an aneurysm site. After the liner and its attached stent are collapsed into low profile configurations, they are compressed and loaded into a sheath in the multi-lumen catheter and sterilized. Then, the catheter/liner system can be delivered in the aneurysm site via the iliac artery with minimum invasivity. It is preferable that the stent is deployed near the neck of the aneurysm to ensure no excess stress is applied on the aneurysm. After the stent is deployed, portion of the liner near the inlet is pressed against the vessel wall by the deployed stent. Then, the sheath of the catheter is removed to expose the to-be expanded liner. During the expansion of the liner, it expands radially toward the aneurysm wall under a hemodynamic force and eventually conforms to the inner surface of the aneurysm wall. After the inner surface of the aneurysm is completely covered by the liner, the liner is inflated by injecting filler through a feeding lumen in the catheter and a feeding tube. The status of inflation is closely monitored by the radiopaque markers on the surface of liner. Excess blood in the aneurysm escapes via the iliac arteries without placing additional stress on the aneurysm wall. Because the outer wall of the liner is already conformed to the inner surface of the aneurysm wall, the injected filler actually moves the inner wall of the liner away from the aneurysm wall. After the pre-determined liner thickness is reached, the feeding tube is removed from the liner. The filler in the liner is then encapsulated in the liner. A second expandable element (e.g. proximal balloon) is positioned and deployed at the outlet junction between the liner and the vessel to ensure the patency of flow conduit during the inflation of the liner. After the filler is hardened, the balloons are collapsed and retrieved from the patient's body. Optionally, a stent or a membrane covered stent is placed at junction between the liner and the vessel wall to ensure seal.

In another deployment method of this invention for treating patient with aneurysm close to the bifurcation (iliac artery), a delivery catheter is used to deliver the stent attached liner in the aneurysm. Expandable element such as a distal balloon can be used in this particular deployment method. The distal balloon is positioned near the distal end of the multi-lumen catheter. In the collapsed configuration, a distal stent and a portion of the liner is placed on top of the distal balloon. After the liner and distal stent are collapsed into low profile configurations, they are compressed and loaded into a sheath in the delivery catheter and sterilized. Then, the catheter/liner system can be positioned in an aneurysm site via the iliac artery with minimum invasivity. It is preferred that the distal stent is deployed near the neck of the aneurysm to ensure no excess stress is applied on the aneurysm. After the distal stent is deployed, portion of the liner is pressed against the vessel wall by the deployed stent. Then, the sheath of the catheter is removed to expose the to-be inflated liner. The liner expands radially toward the aneurysm wall by a hemodynamic force and eventually conforms to the inner surface of the aneurysm wall. After the inner surface of the aneurysm wall is completely covered by the liner, both iliac stents are deployed in iliac arteries respectively to ensure seal at junctions between the liner and iliac arteries. Then a balloon catheter is inserted in the liner via the left iliac artery. Once it is in position, a second balloon on the distal end of the balloon catheter is inflated with saline. At about the same time, a proximal balloon on the delivery catheter is also inflated by saline. Both balloons are used to ensure patency of the flow conduit when the liner is inflated. As the liner is inflated by injected filler, the status of inflation is monitored by radiopaque markers on the liner. Because the outer wall of the liner is already conformed to the inner surface of aneurysm wall, the injected filler actually moves the inner wall of liner away from aneurysm wall. After the appropriate liner thickness is reached, feeding tube is pulled away from the liner and is retrieved. The filler is encapsulated in the liner providing protection to the aneurysm wall. Finally, all balloons are deflated, and the delivery catheter is retrieved from the patient's body leaving the inflated liner in aneurysm. This invention is particularly suitable for treating patients with abdominal aortic aneurysms near the iliac bifurcation.

According to this invention, many suitable filler materials can be used to fill the inflatable multiple walls liner. It is required that the filler is a fluid during the inflating process to pass through the delivery catheter, the feeding tube and finally the inflatable chamber. This fluid filler can be gel, glue, foam, slurry, water, blood, saline, etc. The preferable filler material is a polymer, an oligomer or a monomer which can harden after injection in the liner. The hardening of these materials can be triggered by either physical or chemical means. Chemical means include curing, cross linking, polymerization, etc. The physical means often involve change in temperature, light, electricity, pH, ionic strength, concentration, magnetic field, etc. After the filler is hardened, the liner can provide additional strength to the aneurysm wall and maintain the shape of the liner to ensure close contact with the inner surface of aneurysm. Alternatively, the filler is not hardened and remains soft after it is injected into the inflatable multiple walls liner. This relatively soft layer will serve as a cushion layer against the surface of the aneurysm.

In another embodiment according to the present invention, a bioactive or a pharmaceutical agent is incorporated into the filler. The bioactive or pharmaceutical agent can be mixed with the filler before injection in the liner. After the deployment of liner in the aneurysm, the agent diffuses into the aneurysm wall and treats the damage in the vessel. Because the liner of this invention is in close contact with the aneurysm wall, the agent can reach the aneurysm wall without being diluted by the blood if the agent is delivered systematically by injection. Many bioactive or pharmaceutical agents can be used to treat aneurysm. Drugs that inhibit matrix metalloproteinases, inflammation or other pathological processes involved in aneurysm progression, can be incorporated into the filler to enhance wound healing and/or stabilize and possibly reverse the pathology. Drugs that induce positive effects at the aneurysm site, such as growth factor, can also be delivered with the filler and the methods described herein. Alternatively, the bioactive or pharmaceutical agent can be coated on the outer surface of the liner directly against the aneurysm wall.

In another embodiment of the present invention, the surface of the liner is treated with fibril, coating, foam or surface texture enhancement. These coatings or surface treatment can increase the surface area on the outer wall of the liner and promote tissue or cell to grow onto the outer wall of the liner. The attached cells or tissue on the wall can enhance the bonding and seal between the vessel wall and the liner. In addition to enhanced bonding, appropriate surface coating or texture can also promote the formation of thrombosis and increase the seal between the liner and the aneurysm wall.

There are several benefits to treat aneurysm with this present invention. 1. The inflatable multiple walls liner strengthens the aneurysm wall and prevents the rupture of aneurysm by reducing the hemodynamic pressure on the aneurysm wall. 2. The collapsed liner is flexible so that it can be loaded in a catheter and access the aneurysm site with minimum invasivity. 3. The flexibility of the liner and the hemodynamic force allow the liner to conform to the inner surface of the aneurysm wall. After the filler in the liner is hardened, the liner will be “locked” in the aneurysm without endoleak or migration. 4. Less filler material is required to cover the inner surface of the aneurysm wall. The resulting liner is more flexible and compatible with the vessel and adjacent organs. 5. There is no excess amount of stress on the vulnerable aneurysm wall during the deployment of the liner. In order to prevent endoleak and migration, it is essential to have close contact between the outer wall of the liner and the surface of the aneurysm wall. This invention addresses the drawbacks of prior arts and allows the liner to conform to the aneurysm wall without placing excess stress on the fragile aneurysm wall. As a result, the systems and methods provided by this present invention are safer than methods disclosed in prior arts. 6. The durability of the liner is better than the stent graft because there is no untreated space, which is prone to endoleak between the liner and aneurysm wall. 7. The present invention can enhance the adhesion of the liner to the aneurysm wall further reducing the risk of liner migration and endoleak. 8. This invention enables the use of bioactive or pharmaceutical agents in the filler to treat aneurysm without dilution. The pathological processes involved in aneurysm progression can be stabilized and possibly be reversed.

### BRIEF DESCRIPTION OF THE FIGURES

FIGS. 1a-c depict the cross sectional views of an aneurysm to be filled by a fill structure as disclosed by the prior arts.

FIGS. 2a-c depict the cross sectional views of an aneurysm which is protected by an inflatable multiple walls liner as described in one embodiment according to the present invention.

FIG. 3a depicts an exterior view of a multiple walls liner as described in one embodiment according to the present invention.

FIG. 3b depicts a cross sectional view of a multiple walls liner as described in FIG. 3a according to the present invention.

FIG. 3c depicts a cross sectional view of a multiple walls liner (as described in FIGS. 3a-b) that has been inflated by filler according to the present invention.

FIG. 4a depicts enlarged cross sectional view of a multiple walls liner as described in FIGS. 3a-b in an embodiment of the present invention.

FIG. 4b depicts enlarged cross sectional view of a multiple walls liner (as described in FIG. 4a) that has been inflated by filler according to the present invention.

FIG. 4c depicts enlarged cross sectional view of another multiple walls liner that has been inflated by filler according to the present invention.

FIG. 5 depicts a cross sectional view of a multiple walls liner as described in one embodiment according to the present invention.

FIG. 6a depicts enlarged cross sectional view of a multiple walls liner as described in FIG. 5 in one embodiment according to the present invention

FIG. 6b depicts enlarged cross sectional view of a multiple walls liner (as described in FIG. 6a) that has been inflated by filler according to the present invention

FIG. 7a depicts an exterior view of a multiple walls liner as described in one embodiment according to the present invention.

FIG. 7b depicts a cross sectional view of a multiple walls liner as described in FIG. 7a according to the present invention.

FIG. 7c depicts a cross sectional view of a multiple walls liner (as described in FIG. 7a) that has been inflated by filler according to the present invention.

FIG. 8a depicts enlarged cross sectional view of a multiple walls liner as described in FIG. 7a in an embodiment of the present invention.

FIG. 8b depicts enlarged cross sectional view of a multiple walls liner (as described in FIG. 8a) that has been inflated by filler according to the present invention.

FIG. 8c depicts enlarged cross sectional view of another multiple walls liner that has been inflated by filler according to the present invention.

FIG. 9 depicts an exterior view of an inflatable channel according to an embodiment of the present invention.

FIG. 10a depicts an exterior view of a multiple walls liner as described in one embodiment according to the present invention.

FIG. 10b depicts a cross sectional view of the multiple walls liner as described in FIG. 10a according to an embodiment of the present invention.

FIG. 10c depicts a cross sectional view of a multiple walls liner (as described in FIG. 10a) that has been inflated by filler according to the present invention.

FIG. 11a depicts a cross sectional view of a multiple walls liner as described in one embodiment of the present invention.

FIG. 11b depicts a cross sectional view of the multiple walls liner (as described in FIG. 11a) that has been inflated by filler according to the present invention.

FIGS. 12a-e depict exterior views of inflatable multiple walls liners as described in several embodiments according to the present invention.

FIG. 13a depicts an exterior view of a multiple walls liner as described in one embodiment according to the present invention.

FIG. 13b depicts a cross sectional view of the multiple walls liner (as described in FIG. 13a) that has been inflated by filler according to the present invention.

FIG. 14a depicts an exterior view of a multiple walls liner as described in one embodiment according to the present invention.

FIG. 14b depicts a cross sectional view of the multiple walls liner (as described in FIG. 14a) that has been inflated by filler according to the present invention.

FIGS. 15a-e depict exterior views of several multiple walls liners as described in various embodiments according to the present invention.

FIGS. 16a-b depict cross sectional views of a valve as described in one embodiment according to the present invention.

FIG. 17a depicts an exterior view of a delivery catheter as described in one embodiment according to the present invention.

FIG. 17b depicts a collapsed multiple walls liner mounted upon a delivery catheter as described in one embodiment according to the present invention.

FIGS. 18a-h depict an exemplary deployment sequence of an inflatable multiple walls liner in an aneurysm according to the teachings of the present invention.

FIGS. 19a-h depict an alternate method to deploy an inflatable multiple walls liner in the aneurysm according to the teachings of the present invention.

FIGS. 20a-j depict yet another alternate method to deploy an inflatable multiple walls liner in an aneurysm according to the teachings of the present invention.

### DETAILED DESCRIPTION

Embodiments according to the present invention provide inflatable multiple walls liners and methods useful for protecting an aneurysm and reducing the risk of implantable medical device post-implantation migration and endoleak. More specifically, the inflatable multiple walls liners and methods provide protection to blood vessel walls against rupture especially at the aneurysm site. The inflatable multiple walls liners also have the advantages of minimizing post-implantation device migration and post-implantation endoleak following liner deployment at an aneurismal site.

For convenience, the devices, compositions and related methods according to the present invention discussed herein will be exemplified by using inflatable multiple walls liner intended to treat abdominal aorta aneurysms or Thoracic aortic aneurysms. However, aneurysms at other locations of the body can be treated with the same devices or methods.

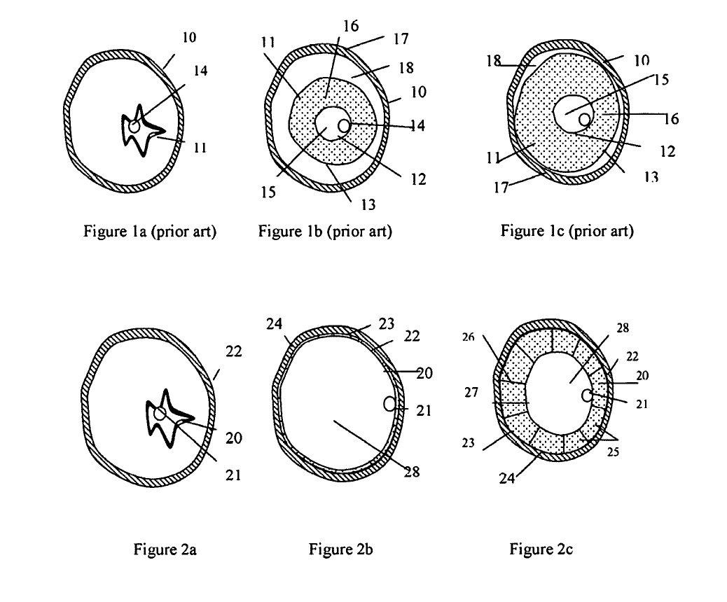

In some embodiments discussed in U.S. patent and patent application Nos. U.S. Pat. Nos. 6,729,356, 5,843,160, 5,665,117, 2004/98096A1 and 2006/212112A1, filler or thrombogenic material is injected into a fill structure in the aneurysm to create hardened material preventing endoleaks. In these methods, a stent graft, a scaffold or a shaping balloon is used to shape the main flow conduit within the fill structure and to prevent the escape of filler. This approach does reduce the chance for accidental embolization in the important vessels. The fill structure is constrained between the aneurysm wall and the stent graft (or scaffold, or a conduit shaping balloon). To ensure conformation to the surface of the aneurysm wall and eliminate the concern of endoleaks and migration, there should be no gap between the fill structure and the aneurysm wall. Insufficient amount of filler will result in gaps between the aneurysm wall and the fill structure and may lead to endoleak and migration. However, too much filler may exert excess circumferential force against the aneurysm wall because of the over-expanded fill structure. This excess circumferential force is risky and may result in aneurysm rupture. With the fill structure discussed in the prior arts, physician cannot determine if the gap has been filled (or not being filled) by the fill structure during the inflation of the fill structure because the potential gap and the aneurysm wall (no contrast agent in them) can not be visualized under Fluoroscope. This uncertainty can place the patient in great risk. As illustrated in the cross sectional view of an aneurysm 10 in FIGS. 1a-1c, fill structure 11 has an inner wall 12 and an outer wall 13. FIG. 1a shows fill structure 11 and injection catheter 14 before inflation. Inner wall 12 defines flow conduit 15 which is usually a tubular shape formed by a stent graft, a scaffold or an inflated tubular balloon (not shown). Filler 16 is injected into fill structure 11 through a lumen in injection catheter 14. The gap between aneurysm wall 17 and flow conduit 15 needs to be totally filled by filler 16 to have good conformation to aneurysm wall 17. As shown in FIG. 1b, injected filler 16 inflates fill structure 11 and expands outer wall 13 radially toward aneurysm wall 17 because flow conduit 15 is already defined by a tubular stent graft or a shaping balloon (not shown). Insufficient amount of filler 16 may lead to a gap 18 between aneurysm wall 17 and outer wall 13 of fill structure 11 as shown in FIG. 1c. However, physician can not visualize gap 18 (no contrast agent) or aneurysm wall 17 under Fluoroscope. On the other hand, too much filler 16 may exert excess circumferential force against aneurysm wall 17. As a result, physician has to “guess” if sufficient filler 16 is injected into fill structure 11.

The present invention addresses the issues with current therapies by providing methods and systems to reduce the likelihood of migration, endoleak and rupture at aneurysm sites. The system comprises an inflatable multiple walls liner which is larger or the same size as the aneurysm to be treated. Referring now to FIGS. 2a-2c, FIG. 2a shows inflatable multiple walls liner 20 and injection catheter 21 before inflation in a cross sectional view of an aneurysm 22. During expansion of liner 20, outer wall 23 of liner 20 expands radially toward and conforms to the inner surface of aneurysm wall 24 by a hemodynamic force as shown in FIG. 2b. Inflation of liner 20 in aneurysm 22 is done by injecting fluid filler 25 through a filling lumen in catheter 21. Because of connectors 26 between the walls 23, 27, inner wall 27 of liner 20 expands in a restrained fashion and defines flow conduit 28 as shown in FIG. 2c. In the present invention, the close contact between the inner surface of aneurysm wall 24 and outer wall 23 of liner 20 is a result of flexible walls 23, 27 and the radial expanding force provided by the hemodynamic force. It is not necessary to fill the whole aneurysm 22 in order to achieve close contact between the inner surface of aneurysm wall 24 and outer wall 23 as disclosed in prior arts. Additional filler 25 in liner 20 expands inner wall 27 toward flow conduit 28 in a restrained fashion and increases the thickness of liner 20 without exerting excess circumferential force against aneurysm wall 24, and without occluding flow conduit 28. In this and in all examples that follow, because of connector 26, the total amount of filler 25 required in order to successfully “exclude” the weakened aneurysm wall 24 from the hemodynamic forces of the aorta is significantly less than that required by the prior art. Less filler 25 which can potentially interfere with vessel remodeling and surrounding organ function following the procedure is required. Further, all filler 25 is securely retained within liner 20, preventing risk of migration of filler 25. Still further, because inflatable liner 20 is conforming to the usually complex topography of the inner surface of the aneurysm 22, inflated liner 20 is “locked” in the aneurysm 22 with minimum chance for migrating out of its designated location and provides reinforcement to the weak aneurysm wall 24. As a result, the system and method described in the present invention are both safe and robust.

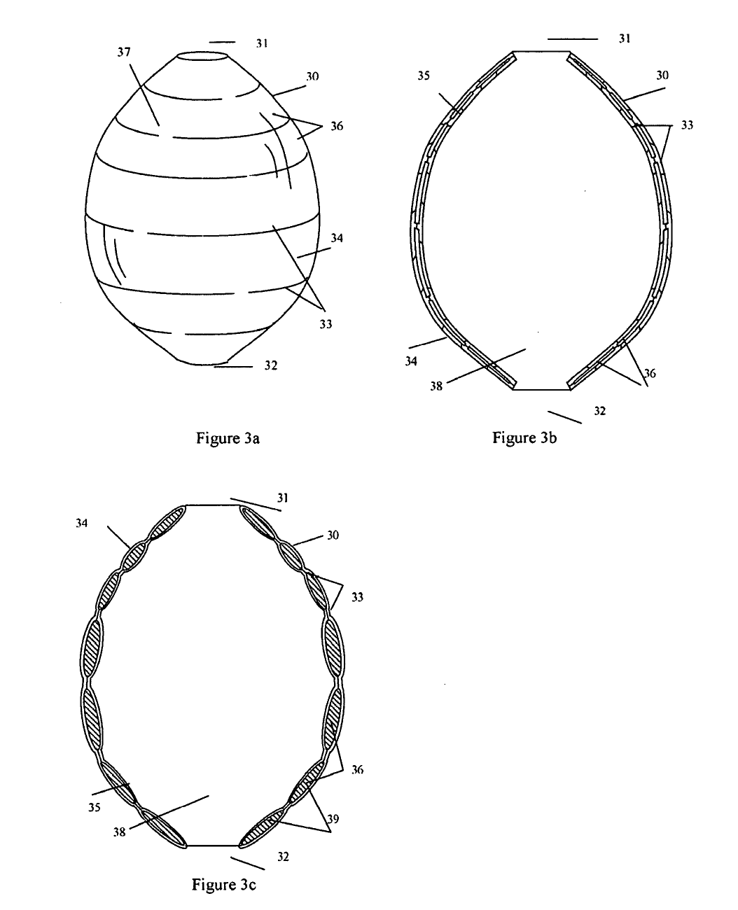

In the present invention, as illustrated in FIG. 3a, inflatable multiple walls liner 30 has the general appearance of a hollow pouch with two openings 31 and 32. Connectors 33 link outer wall 34 and inner wall 35 together at various locations to form interconnected inflatable chambers 36 in liner 30 as shown in FIG. 3b. Discontinuity 37 of connector 33 allows fluid communication between inflatable chambers 36. The embodiment of this invention with two openings 31, 32 is particularly suitable for treating patients with Thoracic aortic aneurysm (TAA), aneurysms in the peripheral arteries, or abdominal aortic aneurysms (AAA) with some distance from the iliac bifurcation.

The materials used for walls 34, 35 are flexible and significantly inelastic so that walls 34, 35 can conform to the inner surface of the aneurysm wall. The materials are biocompatible and not permeable to the fluid filler. Each wall 34, 35 can be made from the same or a different biocompatible material. Typical biocompatible materials are Dacron®, Nylon, PET, PE, PP, FEP, PU or ePTFE film or sheet. They can be extruded, woven, blow molded or molded into a thin sheet or film. The processing technologies are well known to one skilled in the art of film or sheet processing. The thin sheet or film may be stitched, glued, bonded or directly molded into the desired pouch shape.

As illustrated in a cross sectional view of liner 30 in FIG. 3b, inner wall 35 and outer wall 34 are connected by a least one connector 33 at selected locations between walls 34, 35 to form one or more inflatable chamber 36 to be filled by fluid filler (not shown). A least one inflatable chamber is required in each inflatable multiple walls liner. Many different connectors can be used in the present invention. Some examples of connectors include, but are not limited to, a strip, a string or a direct bond, such as glue bond, weld bond, heat bond, etc. Each inflatable multiple walls liner can utilize one particular connector or a mix of several different types of connectors to achieve the desired performance. The type of connector chosen also determines the thickness of the liner after inflation. If a strip or a string is used, its span (length) between the walls defines the thickness of the liner. However, if a direct bond is utilized, the thickness of the walls generally defines the thickness of the liner at the point of bonding. The material used for the connector can be the same material used for the walls with significant inelasticity to avoid excess stretching during inflation. The extent of the connection by the connector between the walls can be a single point, an area, or a line. Combined with the walls, the arrangement and the type of connection between the walls define the shape of the inflatable chamber to be filled by the filler. As an example, direct bonding is used as connector 33 to bond two walls 34, 35 together in liner 30.

FIG. 3b shows a cross sectional view of liner 30 with inner wall 35 and outer wall 34. Flow conduit 38 is defined by inner wall 35 and two openings 31, 32. FIG. 3c is a cross sectional view of liner 30 after fluid filler 39 is introduced into liner 30 to fill inflatable chambers 36 and eventually the whole liner 30. After deployment in the aneurysm, liner 30 would have the shape defined by the inner surface of the aneurysm wall. The blood flow conduit 38 would have a shape determined by the inner surface of the aneurysm wall and the thickness of inflated liner 30.

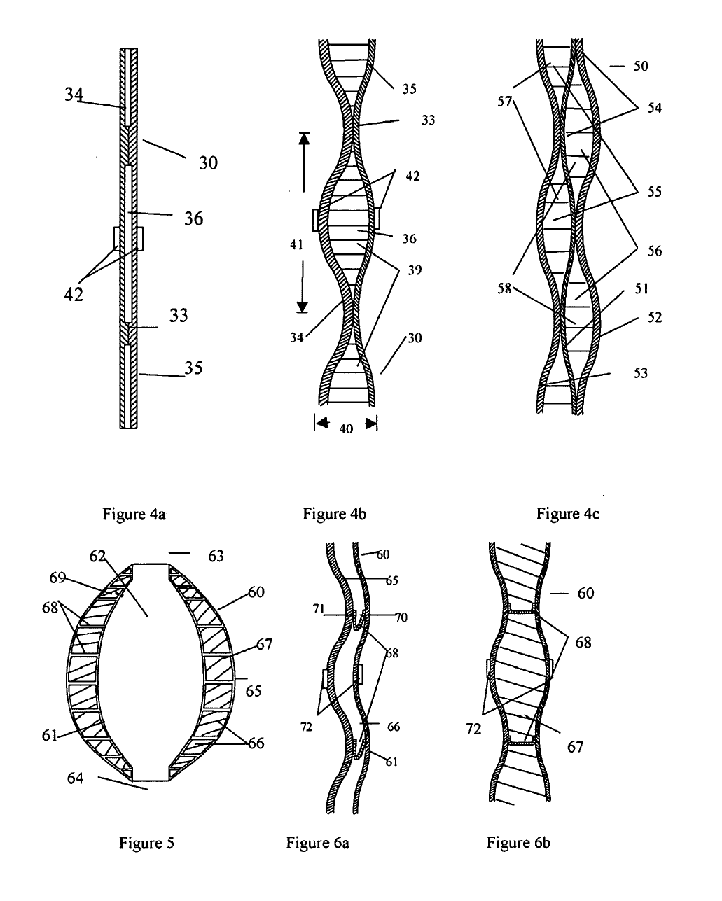

FIGS. 4a-c are the enlarged cross sectional views of walls 34, 35 of exemplary inflatable multiple walls liner 30 (in FIGS. 3a-c) according to the teaching of this invention. In FIG. 4a, outer wall 34 of liner 30 is bonded to inner wall 35 at connectors 33 forming an inflatable chamber 36 to be filled by fluid filler 39 (not pictured). FIG. 4b describes the cross sectional configuration of the same liner 30 after inflatable chamber 36 is inflated by filler 39. Various bonding techniques such as glue bond, weld bond, heat bond, etc. can be used at a plurality of locations between walls 34, 35. As described above, the extent of the bond can be a dot, an area, a line, a dotted line or a combination of the above.

As illustrated in FIG. 4b, the thickness of liner 30 and inflatable chamber 36 is one of the factors determining the flexibility of liner 30. If thickness 40 is broad, liner 30 and inflatable chamber 36 have a lower flexibility after inflation. If thickness 40 is slim, liner 30 and inflatable chamber 36 have a higher flexibility after inflation. Additionally, distance 41 between connectors 33 is another factor affecting the flexibility of liner 30 and inflatable chambers 36. Liner 30 and inflatable chambers 36 are usually thinner at connectors 33 where walls 34, 35 join together (as illustrated in FIG. 4b). Liner 30 and inflatable chambers 36 are usually thicker where it is further away from connector 33 and walls 34, 35 are not constrained by connector 33 and expand outwards. If distance 41 is long, liner 30 and inflatable chamber 36 would be broad with a lower flexibility after inflation. If distance 41 is short, liner 30 and inflatable chamber 36 is slim with a higher flexibility after inflation. In this invention, it is preferable that liner 30 and inflatable chamber 36 are thinner (either by shorter connector 33, shorter distance between connectors 33, or both) near openings 31, 32 of main flow conduit 38. This will increase liner's flexibility to comply with patient's anatomy near the openings 31, 32 to achieve the optimum seal. On the other hand, liner 30 and inflatable chamber 36 can be thicker in the middle of the aneurysm for additional strength. The thicker liner 30 and inflatable chamber 36 can be achieved by a longer connector 33 or a longer distance 40 between connectors 33. The longer connector 33 can be achieved by using connector such as a strip or a string between walls 34, 35.

Connectors 33 serve as a “soft point” to enhance the flexibility of liner 30 after liner 30 is inflated. As described above, liner 30 and inflatable chambers 36 is usually thinner at connector 33 forming a soft point to allow liner 30 to bend easier at that location and relieves any potential stress which may result from body's movement.

As discussed before, the aneurysm wall is usually weak and prone to rupture, it is critical to be able to monitor the progress of liner inflation to achieve success treatment on the aneurysm wall. Radiopaque markers 42 are placed on both inner 35 and outer 34 walls of liner 30 as shown in FIG. 4a-b. As liner 30 is inflated by filler 39, thickness 40 of liner 30, which can be measured between radiopaque markers 42 under a fluoroscope, is increasing until the pre-determined liner thickness 40 is reached. This embodiment of the present invention provides physicians a safe tool to know directly the status of the liner deployment and inflation without “guessing” compared methods suggested by prior arts.

Alternatively, more than two walls can be used to form the inflatable multiple walls liner as shown in a cross sectional configuration of liner 50 in FIG. 4c. A third wall 51 is laminated between inner wall 52 and outer wall 53. Together with the walls 51, 52, 53, alternating connectors 54 between these walls 51, 52, 53 form a plurality of inflatable chambers 55, 56. Inflatable chambers 55 and 56 can be filled by the same filler or different filler with different curing time or hardness to achieve the optimum protection of the aneurysm. For example, inflatable chambers 55 adjacent to outer wall 53 may be filled with softer filler 57 for better cushion with the aneurysm wall. Inflatable chambers 56 adjacent to inner wall 52 may be filled with harder filler 58 for better support for the flow conduit that will be defined by inner wall 52 within the vessel (not pictured).

In another embodiment according to the teaching of this invention, a strip-like connector may be used to link inner and outer walls to form interconnected inflatable chambers in an inflatable multiple walls liner. As illustrated in the cross sectional view of liner 60 in FIG. 5, inner wall 61 defines blood flow conduit 62 between first opening 63 and second opening 64. The space between outer wall 65 and inner wall 61 comprises at least one inflatable chamber 66 to be filled by injected filler 67. Each inflatable chamber 66 is defined by inner wall 61, outer wall 65 and strip connectors 68. Valve 69 is used to inject filler 67 into inflatable chamber 66. Fluid communication is achieved by flow ducts (not shown) among inflatable chambers 66. After deployment in the aneurysm of a subject, multiple walls liner 60 would have the shape defined by the morphology of the inner surface of the aneurysm wall. Blood flow conduit 62 may have a shape depending upon the actual morphology of the inner surface of the aneurysm wall and the thickness of liner 60. This invention is particularly suitable for treating patients with Thoracic aortic aneurysm (TAA), aneurysms in the peripheral arteries, or abdominal aortic aneurysms (AAA) with some distance from the iliac bifurcation.

FIGS. 6a-b are enlarged cross sectional views of an exemplary inflatable multiple walls liner 60 with strip connectors as shown in FIG. 5. As illustrated in FIG. 6a, ends 70, 71 of strip connector 68 are bonded to inner wall 61 and outer wall 65 respectively. An inflatable chamber 66 is defined by walls 61, 65 and connectors 68. Radiopaque markers 72 are attached to inner wall 61 and outer wall 65 and are visible under fluoroscopy. After being inflated by filler 67, inflatable chamber 66 expands outwardly, and the extent of its expansion is limited by strip connectors 68, as shown in FIG. 6b. The increase in distance between radiopaque markers 72 indicates the extent of inflation and can be monitored by physician under fluoroscope during deployment of liner 60 in a subject.

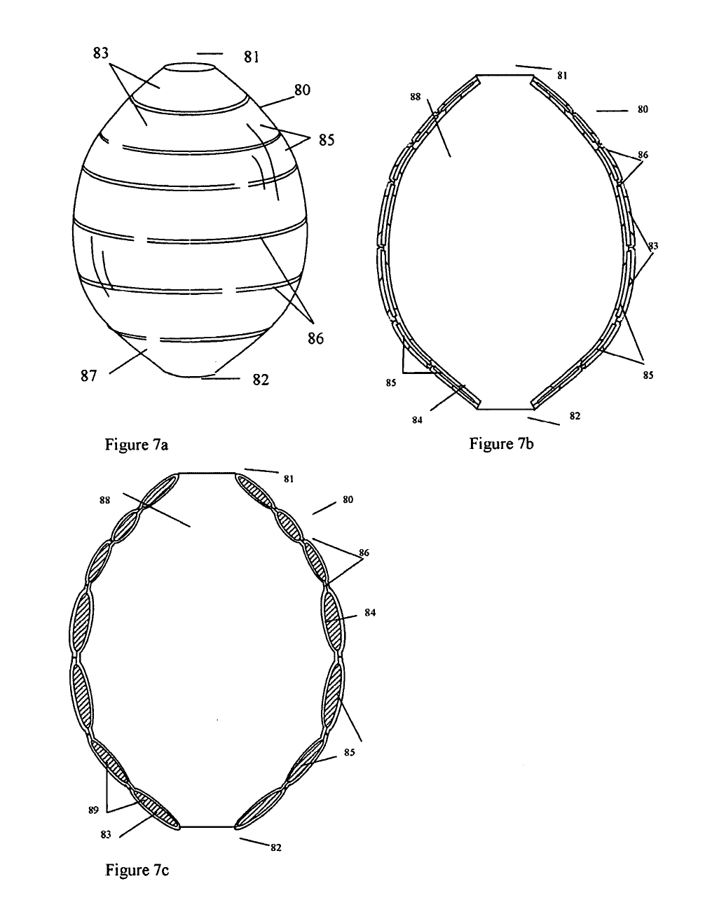

In another embodiment of this invention, the inflatable liner is formed by attaching a plurality of inflatable patches on a pouch shape wall. FIG. 7a illustrates an exemplary inflatable liner 80 with two openings 81, 82. Inflatable patches 83 can be connected to either side of pouch shape wall 84 to form inflatable chamber 85. Various patterns for connector 86 can be used to connect inflatable patch 83 to wall 84. In this example, inflatable patches 83 are connected to the outside of wall 84 circumferentially between two openings 81, 82 herein to form inflatable liner 80. Discontinuity 87 of connector 96 allows fluid communication between inflatable chambers 85. Alternatively, a continuous inflatable patch 83 can be bonded to the outside of wall 84 spirally between two openings 81, 82 to form inflatable liner.

As shown in the cross sectional view of liner 80 in FIG. 7b, inflatable patches 83 are bonded to pouch shape wall 84 and become an outer wall of inflatable liner 80. The bonds between patch 83 and pouch shape wall 84 are connectors 86. Each inflatable chamber 85 is defined by inflatable patch 83 (i.e. outer wall) and pouch shape wall 84 and connectors 86 (i.e. bond). As illustrated in FIG. 7b, wall 84 defines blood flow conduit 88 with a first opening 81 and a second opening 82. FIG. 7c is a cross sectional view of liner 80 after fluid filler 89 is introduced into liner 80 to fill inflatable chambers 85 and eventually the whole liner 80. After deployment in the aneurysm, liner 80 would have the shape defined by the inner surface of the aneurysm wall. Blood flow conduit 88 would have a shape determined by the inner surface of the aneurysm wall and the thickness of inflated liner 80. This embodiment of the present invention is particularly suitable for treating patients with Thoracic aortic aneurysm (TAA), aneurysms in the peripheral arteries, or abdominal aortic aneurysms (AAA) with some distance from the iliac bifurcation.

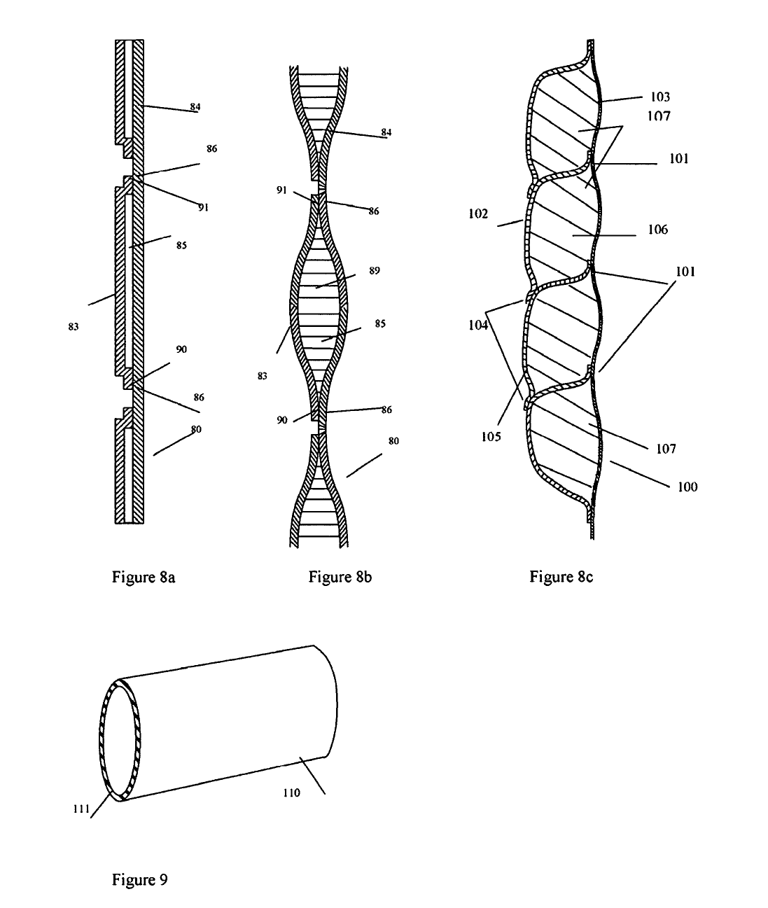

FIGS. 8a-b are the enlarged cross sectional views of liner 80 in FIGS. 7a-c, an inflatable chamber 85 is formed by bonding two edges 90, 91 of an inflatable patch 83 on a pouch shape wall 84. The attachment of inflatable patch 83 on wall 84 and formation of connector 86 can be performed by glue bond, weld bond, heat bond, etc. After inflatable chamber 85 is filled by filler 89, inflatable patch 83 and wall 84 expands outwards to increase the thickness of inflatable chamber 85 and liner 80 as depicted in FIG. 8b. Alternatively, inflatable patches 83 can be attached on either side of pouch shape wall 84.

Alternatively, portion of inflatable patch can be placed on top of adjacent inflatable patch. A cross sectional view of liner 100 is depicted in FIG. 8c, while one edge 101 of inflatable patch 102 is bonded to pouch shaped wall 103, the other edge 104 of inflatable patch 102 is bonded to adjacent inflatable patch 105 forming inflatable chamber 106 to be filled by filler 107. The inflatable patch 102, 105 becomes the outer wall of liner 100, and pouch shape wall 103 becomes the inner wall. A portion of inflatable patches 102, 105 becomes connectors between inner wall 103 and outer wall of liner 100. After filler 107 is injected in liner 100, a relatively consistent liner thickness can be achieved by this approach.

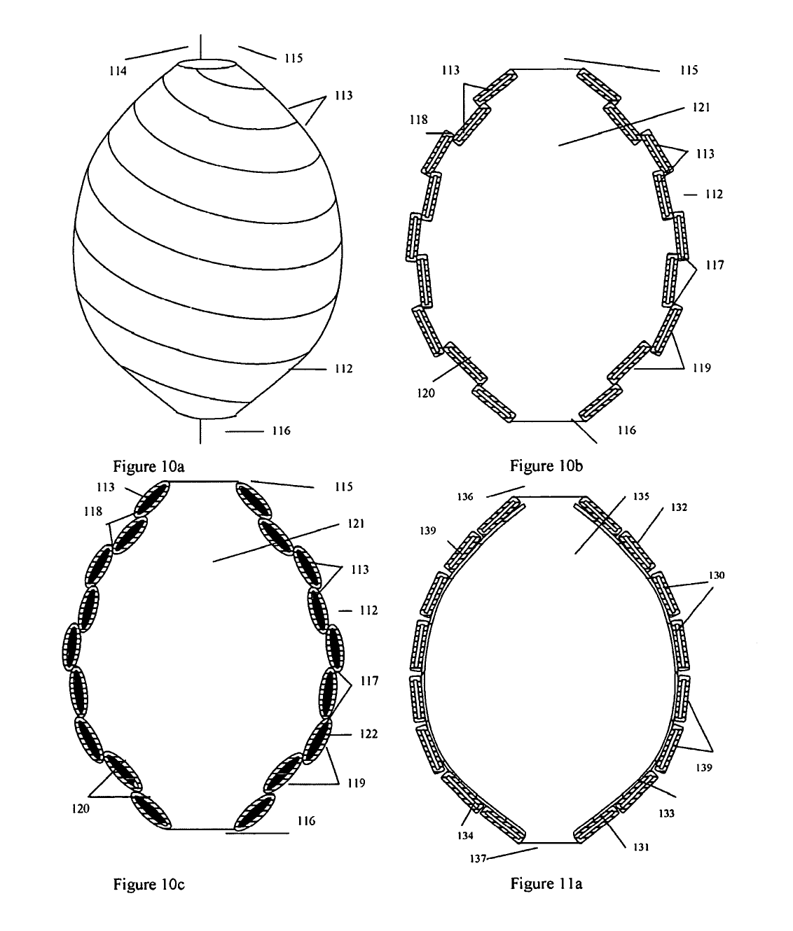

In another embodiment of this invention, inflatable channels are bonded together to form interconnected inflatable chambers of an inflatable multiple walls liner. As shown in FIG. 9, the inflatable channel is a hollow tube 110 having flexible wall 111 which is not permeable to the fluid filler (not shown). Continuing to FIG. 10a, liner 112 comprises a continuous inflatable channel 113 which is arranged spirally about axis 114 extending between opening 115 and opening 116. The pattern of inflatable channel 113 can affect the flexibility and strength of inflatable liner 112. The spiral pattern described herein is one of the exemplary patterns according to the teaching of this invention. As illustrated in FIG. 10b, inflatable channel 113 is bonded together side-by-side at edges 117 of inflatable channel 113 to form connectors 118 and a continuous inflatable chamber 119 as shown in this cross sectional view of line 112. This bonding can be done by heat, weld, glue, etc. Inner wall 120 defines blood flow conduit 121 with a first opening 115 and a second opening 116. FIG. 10c is a cross sectional view of liner 112 after fluid filler 122 is introduced into liner 112 to fill inflatable chambers 119 and eventually the whole liner 112. As discussed above, connector 118 at edge 117 creates a thinner area in liner 112 to enhance its flexibility in the axial direction. Alternatively, instead of spiral pattern described in FIG. 10a, inflatable channels 113 can be bonded side-by-side circumferentially between two openings 115, 116 to form inflatable liner. After deployment in the aneurysm, liner 112 would have the shape defined by the inner surface of the aneurysm wall. Blood flow conduit 121 would have an irregular shape determined by the inner surface of the aneurysm wall and the thickness of inflated liner 112. This embodiment of the present invention is particularly suitable for treating patients with Thoracic aortic aneurysm (TAA), aneurysms in the peripheral arteries, or abdominal aortic aneurysms (AAA) with some distance from the iliac bifurcation.

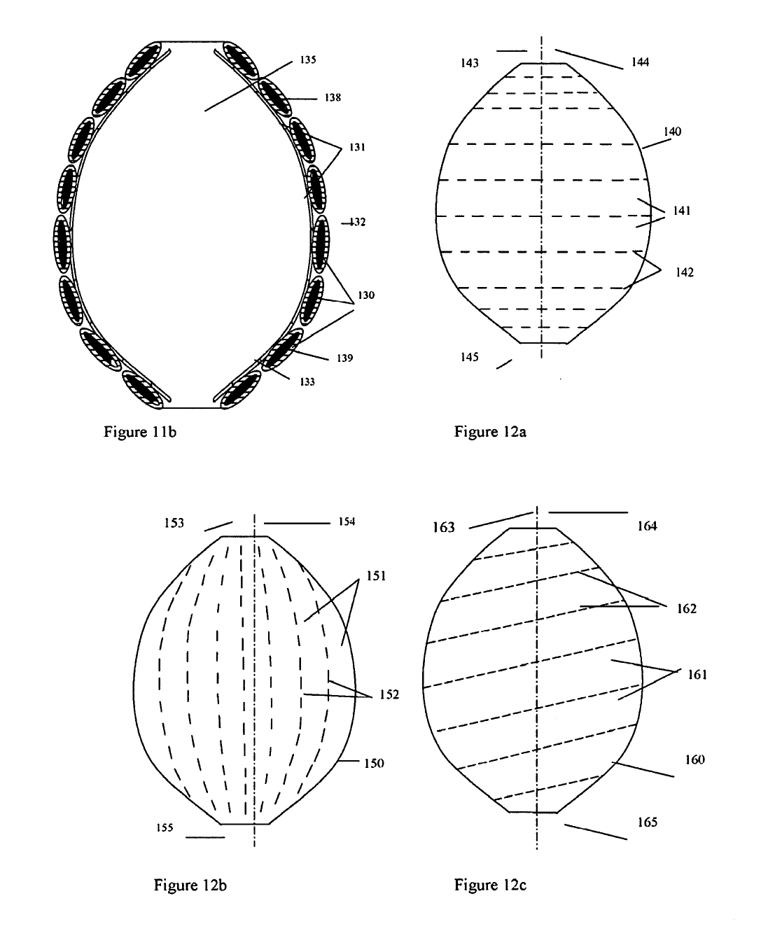

In an alternative method shown in a cross sectional view in FIG. 11a, an continuous inflatable channel 130 can be bonded spirally to either side of a pouch shape wall 131 to form a multiple walls liner 132 with inner wall 133, outer wall 134 and flow conduit 135 between openings 136, 137. FIG. 11b is a cross sectional view of liner 132 after fluid filler 138 is introduced into liner 132 to fill inflatable channels 139 and eventually the whole liner 132. Alternatively, inflatable channels 130 can be bonded to either side of a pouch shape wall 131 circumferentially about an axis extending between openings 136, 137 to form an inflatable liner.

In another embodiment of the present invention, an inflatable multiple walls liner is created by combining inflatable chambers of various forms such as an inflatable patch and an inflatable channel. In yet another embodiment of the present invention, inflatable chambers can be filled with fillers of different stiffness.

As discussed above, the length of connector between the walls and the distance between the connectors determine the thickness and flexibility of the inflatable chamber and liner. Direct bonding between the walls forms a relatively short connector (i.e. the span is merely the thickness of the bond) with thin liner at the bonding. A shorter distance between the connectors with a short connector leads to a liner with a thinner wall. On the other hand, a longer distance between the connectors with a long connector (in the case of using connector such as a strip or a wire) results in a thicker liner. As a result, the thickness and flexibility of the liner can be controlled by selecting the appropriate connector, its distance between the connectors and its connector thickness between the walls.

Additionally, the arrangement, (i.e. pattern), of connectors in the liner is also important in determining the flexibility and strength of the liner. The pattern defines not only the distance between the connectors but also the orientation of the connectors. As discussed above, connectors may result in a thinner area in the liner and serve as a “soft point” for the liner. This characteristic allows the liner to have flexibility in the desired direction to conform to body movement. At the same time, it is also desirable to have a liner with sufficient thickness and strength to protect the aneurysm from rupturing.

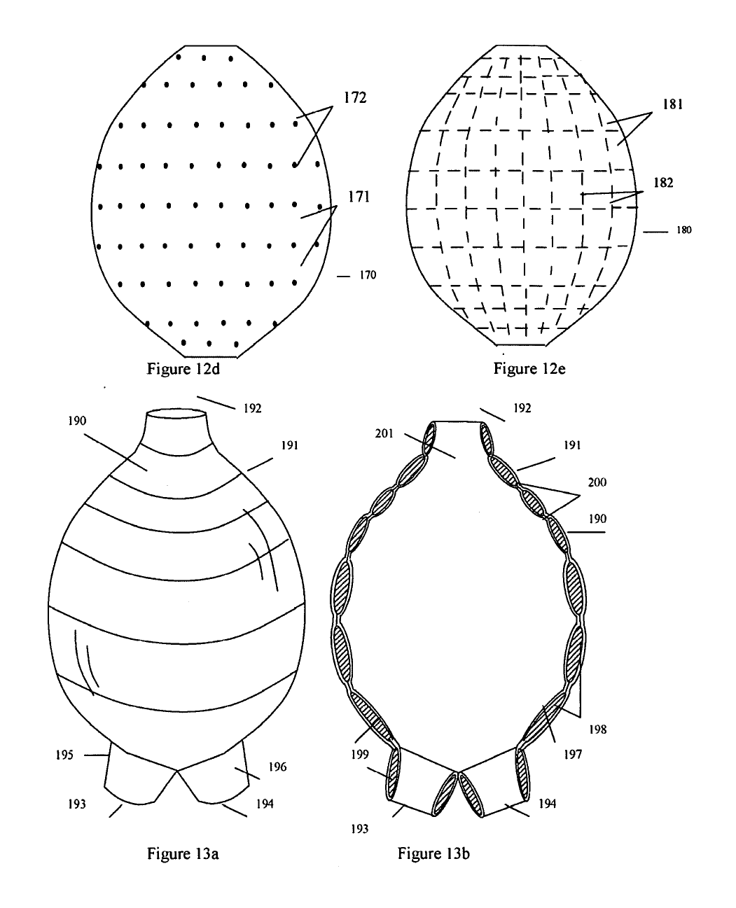

Some exemplary connector patterns are described in FIGS. 12a-e. The dotted lines or points indicate the locations of the connectors in the wall. A strip, a string, a direct bonding or a combination of the foregoing can be utilized to form one or more connectors between the walls. The walls and connectors define inflatable chambers in the respective liners with which they are illustrated. A plurality of flow ducts (not shown) between inflatable chambers allow fluid communication between inflatable chambers in the liners.

As shown in FIG. 12a, inflatable multiple walls liner 140 comprises plurality of inflatable chambers 141 (divided by connectors 142) arranged circumferentially along axis 143 between two openings 144 and 145. This connector pattern provides liner 140 with a high flexibility along axis 143 between two openings 144, 145 and a high circumferential stiffness after liner 140 is inflated. On the other hand, liner 150, shown in FIG. 12b, has plurality of inflatable chambers 151 (divided by connectors 152) arranged along axis 153 between two openings 154 and 155. Due to its connector pattern, liner 150 has a high flexibility circumferentially and a high stiffness along axis 153 after it is inflated. FIG. 12c illustrates a liner 160 with inflatable chambers 161 (divided by connectors 162) encircling axis 163 helically between two openings 164 and 165. This particular connector pattern has a compromised flexibility and stiffness as compared to liners 140 and 150 in both circumferential and axial directions after liner 160 is inflated.

Liners 170 and 180 with connector patterns described in FIGS. 12d-e do not have a particular stiffness or flexibility bias in either circumferential or axial direction. Actually, there is only one inflatable chamber 171 with a plurality of pointed connectors 172 in liner 170 described in FIG. 12d. FIG. 12e illustrates liner 180 with inflatable chambers 181 (divided by connectors 182) with no particular stiffness or flexibility bias in either circumferential or axial direction.

In another embodiment of the present invention, a connector is placed at a needed location to serve as “stress relief” or a “bend point” because of the thinner liner near the connector as discussed above. The circumferential flexibility of liner 140 described in FIG. 12a can be enhanced by introducing connectors in the axial direction as shown in FIG. 12b. These exemplary connector patterns are described herein to demonstrate the ability to achieve a desirable liner flexibility and stiffness by utilizing various connectors, and by varying their orientation, distance between connectors and thickness.

In another embodiment of the present invention, the inflatable multiple walls liner is particularly suitable for lining an aneurysm disposed in close proximity to a bifurcation, such as an aortic aneurysm adjacent to the iliac artery. FIGS. 13a-b are the perspective and cross sectional views of the exemplary liners according to the teaching of this invention. In FIG. 13a, outer wall 190 of liner 191 is flexible, and has three openings 192, 193 and 194. Two openings 193 and 194 leading to the bifurcation are adjacent to each other. There are sleeves 195, 196 connected to openings 193, 194 respectively to enhance the seal between liner 191 and the vessel wall. The space between outer wall 190 and inner wall 197 comprises at least one inflatable chamber 198 filled by injected filler 199 as depicted in the cross sectional view of liner 191 in FIG. 13b. Pluralities of connectors 200 between walls 190, 197 determine the thickness of inflated liner 191. A short length connector (e.g. connector formed via bonding) is used herein as an example. However, a long length connector (e.g. a connector formed via strip or string) can also be used. Inner wall 197 defines blood flow conduit 201 with one inlet 192 and two outlets 193 and 194. Each of the outlets 193 and 194 leads to an iliac artery respectively. After the deployment within the aneurysm, liner 191 will have the shape defined by the morphology of the inner surface of the aneurysm wall. The shape of blood flow conduit 201 will be determined by both the morphology of the inner surface of the aneurysm wall and the thickness of liner 191.

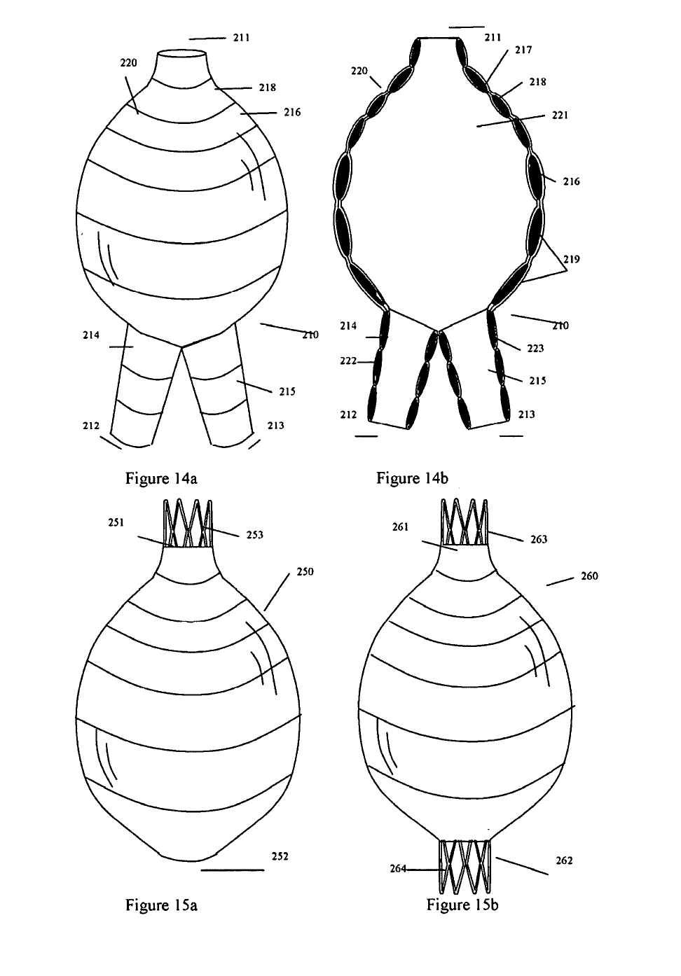

In yet another embodiment of the present invention, the inflatable liner is particularly suitable for lining aneurysm which has extended from aorta to iliac artery. FIGS. 14a-b are the perspective and cross sectional views of the exemplary liners according to the teaching of this invention. Liner 210 is hollow with three openings 211, 212, 213 as shown in FIG. 14a. Two of the openings 212, 213 leading to the bifurcation are adjacent to each other and are configured to mate with an iliac artery respectively. The sleeves 214, 215 extended from openings 212, 213 enhance the seal between liner 210 and the vessel wall and protect aneurysm in the iliac arteries. The space between outer wall 216 and inner wall 217 comprises at least one inflatable chamber 218 filled by injected filler 219 as depicted in the cross sectional view of liner 210 in FIG. 14b. Pluralities of connectors 220 between walls 216, 217 define the thickness of main inflated liner 210. A short connector (i.e. one formed via bonding) is used herein as an example. However, a long connector 220 (i.e. one formed via a strip) can also be used. Inner wall 217 defines the blood flow conduit 221 with one inlet 211 and two outlets 212 and 213. Inflatable bifurcated sleeves 214, 215 have inflatable chambers 222 and 223, which are in fluid communication with inflatable chambers 218 in the main inflatable liner 210 to provide protection to the aneurysm in both the aorta and the iliac arteries. After deployment within the aneurysm, blood flow conduit 221 will have a shape determined by both the inner surface of the aneurysm and the thickness of liner 210.

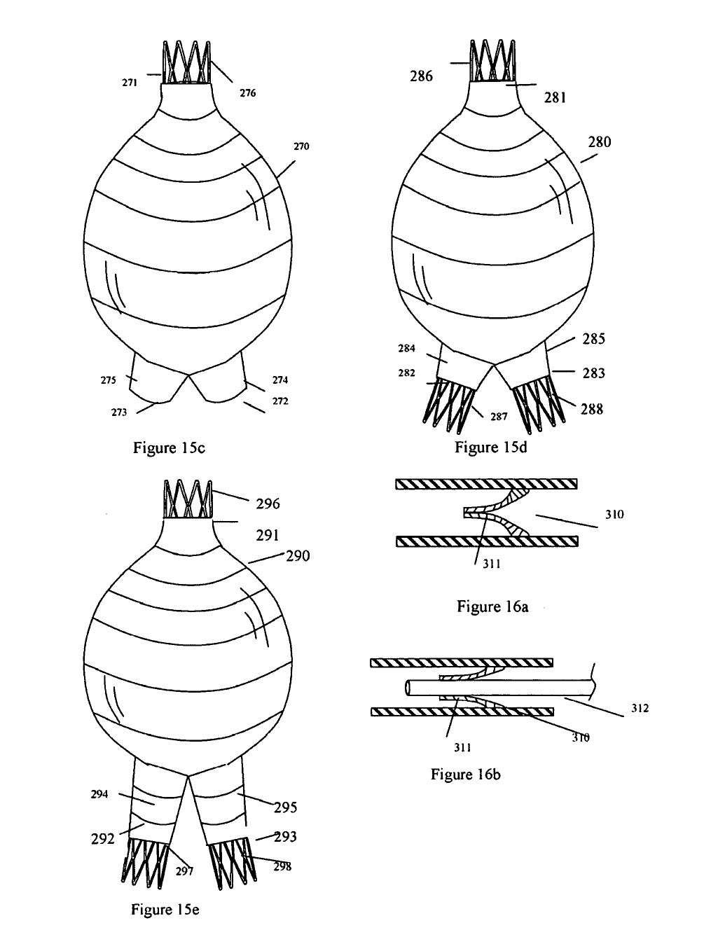

In yet another embodiment of the present invention, at least one stent is permanently fixed to one of the openings of the inflatable liner for anchoring and sealing the liner on the vessel wall. The stent is either self-expandable either or by the outward radial force exerted by another expandable element so that stent can expand and anchor liner to the vessel walls after deployment. Typical biocompatible materials for stent are stainless steel, Nitinol or plastic. FIGS. 15a-15e are the perspective views of the exemplary liners according to the teaching of this invention. As shown in FIG. 15a, liner 250 is hollow with two openings 251, 252. At least one stent 253 is permanently fixed to liner 250 near opening 251. Stent 253 is stitched, glued, or bonded to inflatable liner 250. Alternatively, inflatable liner 260 is hollow with two openings 261, 262 as illustrated in FIG. 15b. One stent 263 is permanently fixed to liner 260 near opening 261. Another stent 264 is permanently fixed to liner 260 near opening 262. Stents 263, 264 are stitched, glued, or bonded to inflatable liner 260. This embodiment of the present invention is particularly suitable for treating patients with Thoracic aortic aneurysm (TAA), aneurysms in the peripheral arteries, or abdominal aortic aneurysms (AAA) with some distance from the iliac bifurcation.

As shown in FIG. 15c, liner 270 is hollow with three openings 271, 272, 273. Two of the openings 272, 273 leading to the bifurcation have sleeve 274, 275 adjacent to each other. Stent 276 is permanently fixed to liner 270 near opening 271 by stitch, glue, or heat bonding. Alternatively, liner 280 is hollow with three openings 281, 282, 283 as illustrated in FIG. 15d. Two of the openings 282, 283 leading to the bifurcation have sleeve 284, 285 adjacent to each other. Stent 286 is permanently fixed to liner 280 near opening 281. One stent 287 is permanently fixed to sleeve 284 leading to one of the iliac arteries. Another stent 288 is permanently fixed to sleeve 285 leading to one of the iliac arteries. This embodiment of the present invention is particularly suitable for treating patients with aneurysms adjacent to bifurcation.

Liner 290 is hollow with three openings 291, 292, 293 as shown in FIG. 15e. Two of the openings 292, 293 leading to the bifurcation have sleeves 294, 295 adjacent to each other. Each of the openings 292, 293 is configured to mate with an iliac artery respectively. Sleeves 294, 295 extended from the openings 292, 293 enhance the seal between the liner 290 and the vessel wall and protect aneurysm in the iliac arteries. Stent 296 is permanently fixed to liner 290 near opening 291. Stents 297, 298 are stitched, glued, or bonded to sleeves 294, 295 leading to iliac arteries respectively. This embodiment of the present invention is particularly suitable for treating patients with aneurysms extended from aorta to iliac artery.

In the practice, physician needs to determine the appropriate liner to use for each patient. With the imaging techniques such as CT scan or MRI, the size and length of the patient's aneurysm can be measured accurately. Then, the physician can select the inflatable multiple walls liner that best fits the patient's aneurysmal anatomy. It is preferable to use a liner with an outer diameter no less than the largest inner diameter of the aneurysm. Because of the flexible wall of the liner and the hemodynamic force, the liner will conform to the inner wall of the aneurysm. By selecting a liner with a larger diameter than the inner diameter of the aneurysm, the extra length of the liner wall will ensure conformation to the aneurysm wall with no gaps between the liner and aneurysm wall.

In another embodiment of the present invention, the inflatable multiple walls liner is inflated via a valve disposed within the liner. As shown in a cross sectional view of valve 310 in FIG. 16a, the valve 310 is in a “closed” position with two leaflets 311 contacting each other. The inserted feeding tube 312 separates leaflets 311 and opens one way valve 310 as illustrated in FIG. 16b.

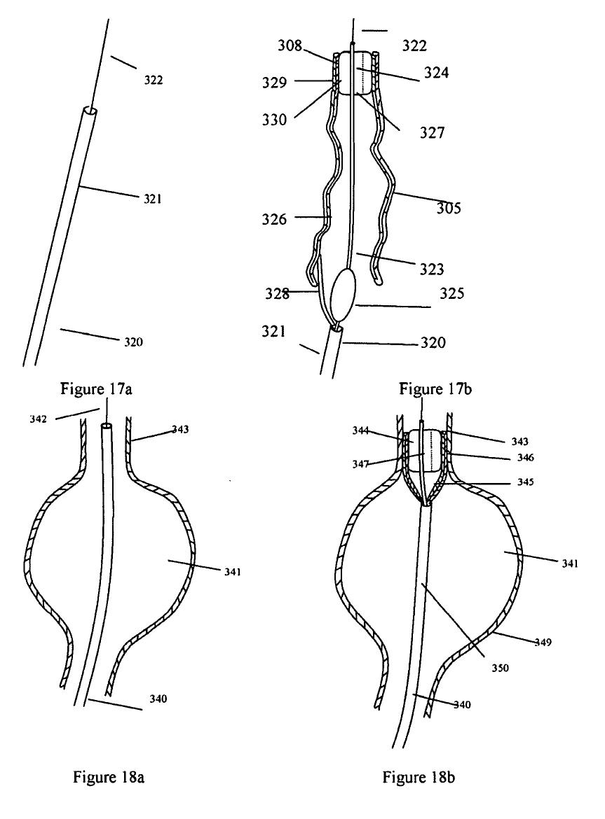

In one embodiment according to the present invention, an inflatable multiple walls liner is pre-loaded into a delivery catheter such as that depicted in FIG. 17a. Delivery catheter 320 has a retractable sheath 321 with compressed liner (not shown) in it. Guidewire 322 can pass through a lumen (not shown) in delivery catheter 320 and used to direct delivery catheter 320 in the body. Within the lumen of catheter 320 is a multilumen catheter 323, as shown in FIG. 17b. Multilumen catheter 323 has a lumen for guide wire 322, a lumen for delivery of filler and lumens for delivery of saline for inflating distal balloon 324 and proximal balloon 325. Distal balloon 324 is positioned at the distal end of multilumen catheter 323 to anchor liner 326 during the deployment procedures. Other than distal balloon 324, various types of expandable elements, such as a self-expandable stent, wire, mesh, etc. can also be used to anchor liner 326 according to the invention. An inflatable distal balloon 324 is used herein as an example. Inflatable distal balloon 324 is preferred to have an annular shape with lumen 327 allowing blood flow through balloon 324 after inflation. Feeding tube 328 that links the filler feeding lumen (not shown) in multilumen catheter 323 is attached to liner 326. In the collapsed configuration, a portion of liner 326 near inlet 329 is mounted on top of distal balloon 324 with inner wall 330 against the surface of distal balloon 324. Feeding tube 328 is inserted in the valve (not shown) within liner 326. Optionally, a second expandable element, such as proximal balloon 325, is placed near the proximal end of multilumen catheter 323. During assembly, after liner 326 and balloons 324 and 325 are collapsed into the low profile configurations, they are radially compressed to fill sheath 321 in the distal end of delivery catheter 320. Liner 326 is covered with retractable sheath 321 and sterilized with various known sterilization methods.

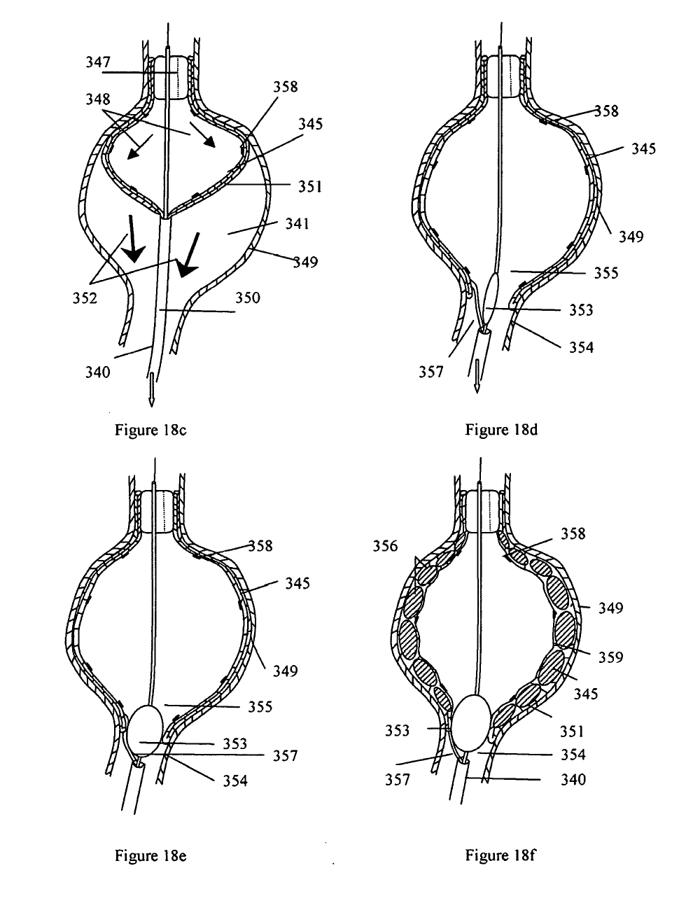

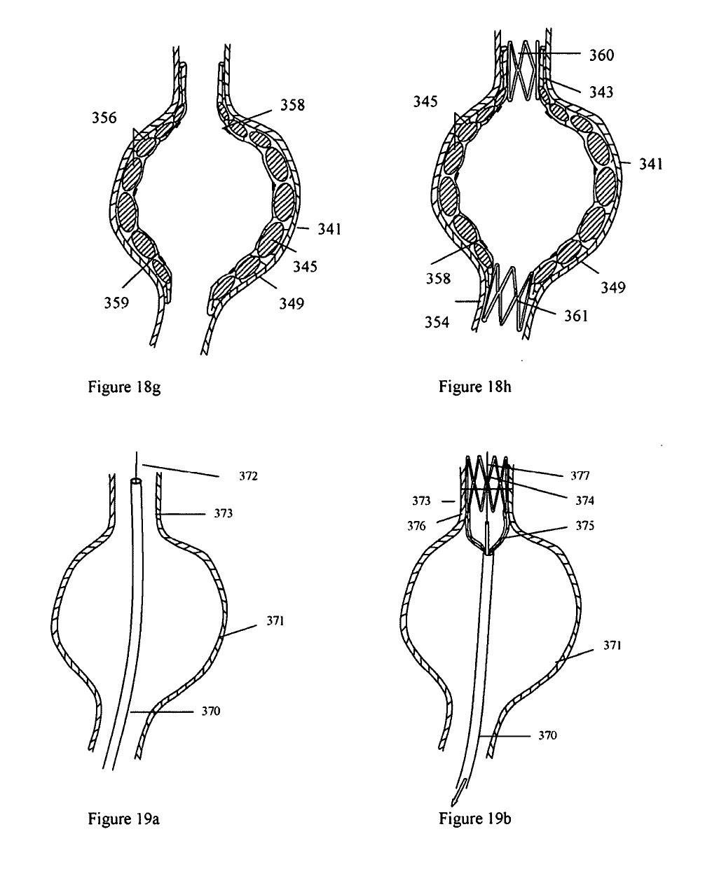

For the preferred deployment method of this invention, a multi-lumen balloon catheter 340 is used to deliver the inflatable multiple walls liner in aneurysm 341 via the iliac artery using a minimally invasive technique. An inflatable multiple walls liner with two openings (as shown in FIG. 3a) is used herein as an example to line aneurysm 341. As shown in FIG. 18a, delivery catheter 340 is guided by guidewire 342 and positioned in the aneurysm 341 with its distal end close to neck 343 of aneurysm 341. It is preferable that distal balloon 344 is deployed near neck 343 of aneurysm 341 to ensure that no excess stress is exerted upon aneurysm 341 as illustrated in FIG. 18b. After distal balloon 344 is inflated, a portion of liner 345 is pressed against vessel wall 346 by the inflated distal balloon 344. At the same time, blood flows through lumen 347 in distal balloon 344 as indicated by arrow 348, in order to expand liner 345 radially toward aneurysm wall 349. As sheath 350 is retrieved to expose liner 345 in sheath 350, the expansion continues until outer wall 351 of liner 345 is against aneurysm wall 349 of aneurysm 341 as depicted in FIGS. 18c-d. As indicated by arrows 352 in FIG. 18c, the existing blood in aneurysm 341 escapes from aneurysm 341 through the gap between catheter 340 and aneurysm wall 349. This procedure is safe because the pressure to expand liner 345 is the same pressure that existed in aneurysm 341 before treatment. No additional stress is placed on aneurysm wall 349 during the liner expansion. After aneurysm wall 349 has been completely covered by liner 345, a proximal balloon 353 is inflated at junction 354 between liner 345 and aneurysm wall 349 as shown in FIG. 18e. Proximal balloon 353 is also preferably of an annular shape and can be on the same catheter 340 or on a separate catheter. Proximal balloon 353 is to ensure that blood flow conduit 355 remains open at junction 354 after the inflation of liner 345. The inflation of liner 345 gives additional strength to liner 345 and protects aneurysm wall 349. It is accomplished by injecting filler 356 into multiple walls liner 345 through a lumen in catheter 340 and feeding tube 357 as shown in FIG. 18f. As liner 345 is inflated, the status of inflation is monitored by radiopaque markers 358 on the surface of liner 345. Alternatively, the status of inflation can be observed if filler 356 becomes radiopaque when additional radiopaque agent has been added to it. Because outer wall 351 of liner 345 already conforms to the inner surface of aneurysm wall 349, the injected filler 356 is actually moving inner wall 359 of liner 345 away from aneurysm wall 349. After the appropriate liner thickness is reached, feeding tube 357 is pulled away from the valve (not shown) in liner 345 and is retrieved. After feeding tube 357 is retrieved, the one way valve is closed, and filler 356 is encapsulated in liner 345. Finally, balloons 344 and 353 are collapsed, and delivery catheter 340 is retrieved from the patient's body leaving inflated liner 345 in aneurysm 341 as shown in FIG. 18g. Optionally, stents 360, 361 or, alternatively, membrane covered stents are placed between liner 345 and aneurysm wall 349 at neck 343 and junction 354 respectively to ensure an adequate seal as shown in FIG. 18h.

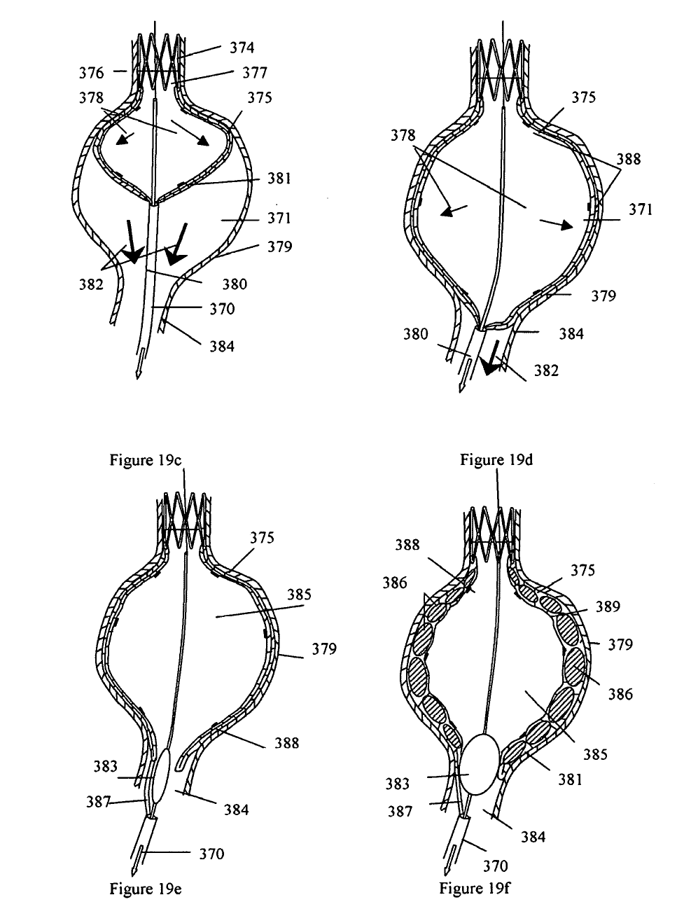

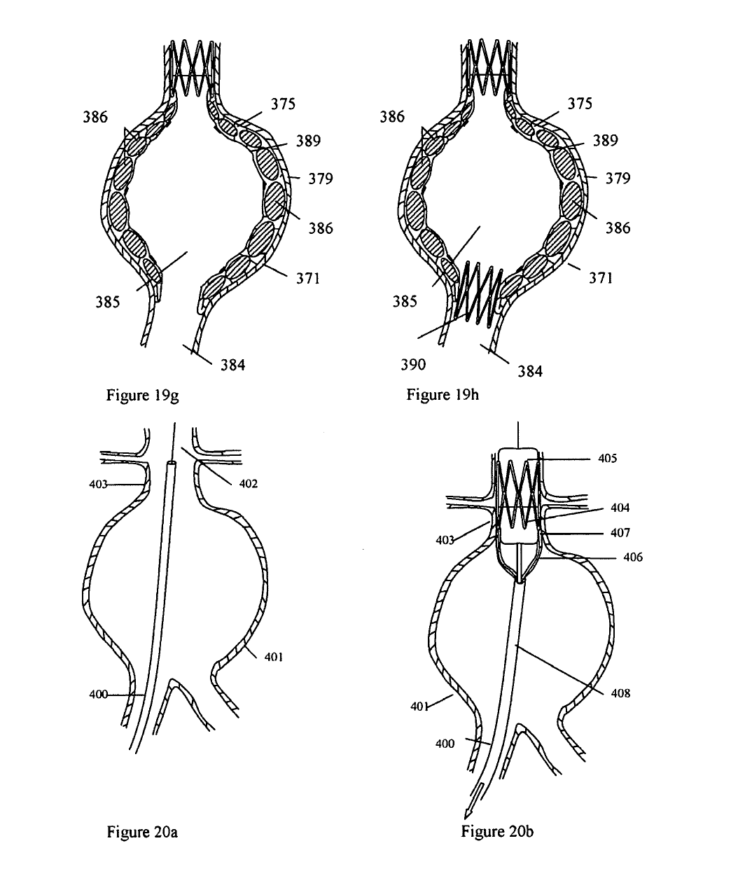

For another preferred deployment method of this invention, a multi-lumen catheter 370 is used to deliver a stent attached inflatable multiple walls liner in the aneurysm 371 via the iliac artery with minimum invasivity. An inflatable multiple walls liner with a stent affixed to one of its openings (as shown in FIG. 15a) is used herein as an example to line aneurysm 371. As shown in FIG. 19a, delivery catheter 370 is guided by guidewire 372 and positioned in aneurysm 371 with its distal end close to neck 373 of aneurysm 371. It is preferable that distal stent 374 is deployed near neck 373 of aneurysm 371 to ensure that no excess stress is exerted upon aneurysm 371 as illustrated in FIG. 19b. After distal stent 374 is deployed, a portion of liner 375 is pressed against vessel wall 376 by the deployed stent 374. At the same time, blood flows through lumen 377 in distal stent 374, as indicated by arrow 378, in order to expand liner 375 radially toward aneurysm wall 379. As sheath 380 is retrieved to expose liner 375 in sheath 380, the expansion continues until outer wall 381 of liner 375 is against aneurysm wall 379 of aneurysm 371 as depicted in FIGS. 19c-d. As indicated by arrows 382 in FIG. 19c, the existing blood in aneurysm 371 escapes from aneurysm 371 through the gap between catheter 370 and aneurysm wall 379. This procedure is safe because the pressure to expand liner 375 is the same pressure that existed in aneurysm 371 before treatment. No additional stress is placed on aneurysm wall 379 during the liner expansion. After aneurysm wall 379 has been completely covered by liner 375, a proximal balloon 383 is inflated at junction 384 between liner 375 and aneurysm wall 379 as shown in FIGS. 19e-f. Proximal balloon 383 is preferably on the same catheter 370 or on a separate catheter. Proximal balloon 383 is to ensure that blood flow conduit 385 remains open at junction 384 after the inflation of liner 375. The inflation of liner 375 gives additional strength to liner 375 and protects aneurysm wall 379. It is accomplished by injecting filler 386 into multiple walls liner 375 through a lumen in catheter 370 and feeding tube 387 as shown in FIG. 19f. As liner 375 is inflated, the status of inflation is monitored by radiopaque markers 388 on the surface of liner 375. Alternatively, the status of inflation can be observed if filler 386 becomes radiopaque when additional radiopaque agent has been added to it. Because outer wall 381 of liner 375 already conforms to the inner surface of aneurysm wall 379, the injected filler 386 is actually moving inner wall 389 of liner 375 away from aneurysm wall 379. After the appropriate liner thickness is reached, feeding tube 387 is pulled away from the valve (not shown) in liner 375 and is retrieved. After feeding tube 387 is retrieved, the one way valve is closed, and filler 386 is encapsulated in liner 375. Finally, balloon 383 is collapsed, and delivery catheter 370 is retrieved from the patient's body leaving inflated liner 375 in aneurysm 371 as shown in FIG. 19g. Optionally, stent 390 or, alternatively, membrane covered stent is placed between liner 375 and aneurysm wall 379 at junction 384 to ensure an adequate seal as shown in FIG. 19h.

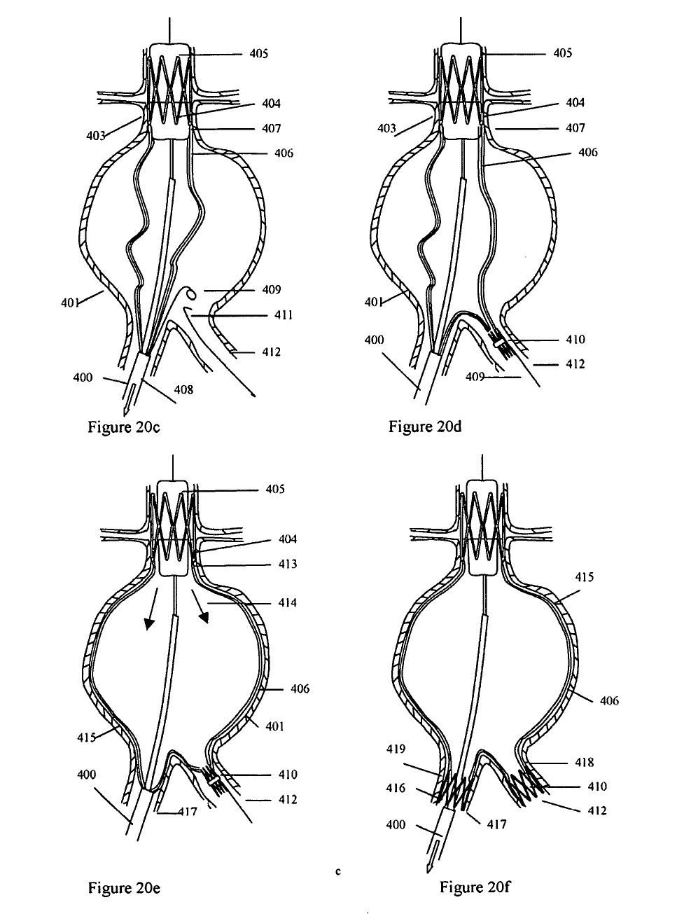

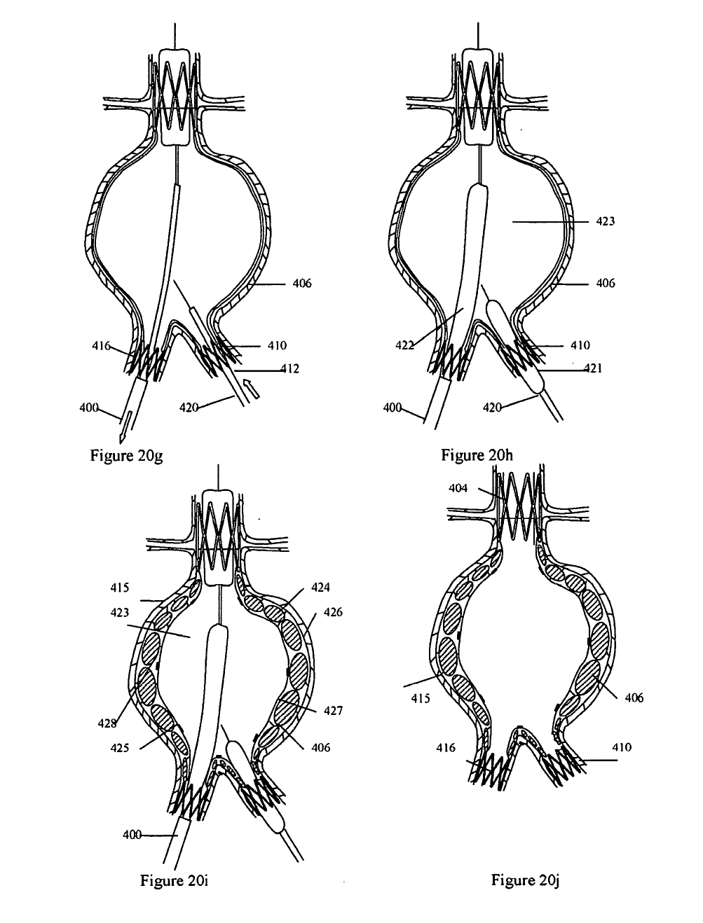

For yet another preferred deployment method of this invention, multi-lumen delivery catheter 400 is used to deliver the stent attached inflatable multiple walls liner in aneurysm 401 via the iliac artery with minimum invasivity. An inflatable multiple walls liner with three stents affixed to its three openings (as shown in FIG. 15d) is used herein as an example to line aneurysm 401 close to the bifurcation. Other exemplary stent attached inflatable multiple walls liners can also be deployed with this method. As shown in FIG. 20a, delivery catheter 400 is guided by guidewire 402 and positioned in aneurysm 401 with its distal end close to neck 403 of aneurysm 401. It is preferable that distal stent 404 is deployed by a distal balloon 405 near neck 403 of aneurysm 401 to ensure that no excess stress is exerted upon aneurysm 401 as illustrated in FIG. 20b. A balloon expandable stent 404 is used herein as an example. Other types of stent such as self expandable stent can also be used in this invention. After distal stent 404 is deployed, a portion of liner 406 is pressed against vessel wall 407 by the deployed stent 404. Then, sheath 408 of catheter 400 is removed to expose the to-be inflated liner 406 and a wire 409 linked to an iliac stent 410 as illustrated in FIG. 20c. Simultaneously, a wire 411 is inserted in aneurysm 401 via left iliac artery 412 to pull wire 409 and iliac stent 410 to the left iliac artery 412 for deployment as shown in FIG. 20d. Distal balloon 405 is then deflated slightly allowing blood flow through space 413 between balloon 405 and distal stent 404 as indicated by arrow 414 in order to expand liner 406. Under this hemodynamic pressure, liner 406 expands radially toward aneurysm wall 415 and eventually conforms to the inner surface of aneurysm wall 415 of aneurysm 401 as depicted in FIG. 20e. This procedure is safe because the hemodynamic force to expand liner 406 is the same force before the procedure. No additional stress is placed on aneurysm wall 415 during the expansion of liner 406.

After aneurysm wall 415 is completely covered by liner 406, both iliac stents 410, 416 are deployed in iliac arteries 412, 417 respectively as shown in FIG. 20f. They are used to ensure seal at junctions 418, 419 between liner 406 and iliac arteries 412, 417. Self expandable stents 410, 416 are used herein as an example. Other types of stents such as balloon expandable stents can also be used in this invention. As shown in FIG. 20g, a balloon catheter 420 is inserted in liner 406 via left iliac artery 412. Once it is in position, balloon 421 on the distal end of catheter 420 is inflated with saline. As shown in FIG. 20h, a proximal balloon 422 on delivery catheter 400 is also inflated by saline. Both balloons 421, 422 are used to ensure patency of flow conduit 423 when liner 406 is inflated. The inflation of liner 406 gives additional strength to liner 406 and protects aneurysm wall 415. It is accomplished by injecting filler 424 into liner 406 through a lumen in catheter 400 and feeding tube (not pictured) as shown in FIG. 20i. As liner 406 is inflated, the status of inflation is monitored by radiopaque markers 425 on the surface of liner 406. Alternatively, the status of inflation can be observed if filler 424 becomes radiopaque when additional radiopaque agent has been added to it. Because outer wall 426 of liner 406 is already conformed to the inner surface of aneurysm wall 415, the injected filler 424 actually moves inner wall 427 of liner 406 away from aneurysm wall 415. A plurality of connectors 428 between inner wall 427 and outer wall 426 defines the thickness of inflated liner 406. After the appropriate liner thickness is reached, feeding tube (not pictured) is pulled away from the valve (not shown) in liner 406 and is retrieved. After feeding tube (not pictured) is retrieved, the one way valve (not shown) is closed, and filler 424 is encapsulated in liner 406 providing protection to aneurysm wall 415. Finally, all balloons 405, 421, 422 are deflated, and delivery catheter 400 is retrieved from the patient's body leaving inflated liner 406 in aneurysm 401 as shown in FIG. 20j. This invention is particularly suitable for treating patients with abdominal aortic aneurysms near the iliac bifurcation.

According to the teaching of this invention, many suitable filler materials can be used to fill the liner. It is required that the filler is a fluid during the inflating process to pass through the catheter and feeding tube and finally the inflatable multiple walls liner. This fluid can be a gel, glue, foam, slurry, water, blood, saline, etc. If blood is used as filler, it can form thrombosis and become hardened in the liner. In this case, a thrombogenic coating on the inner surface of the inflatable chamber can accelerate the formation of thrombus. The preferred filler material is a non-biodegradable material such as polymer, oligomer or monomer which can harden after injection in the liner. The hardening of the non-biodegradable material can be triggered by either physical or chemical means. Chemical means include curing, cross-linking, polymerization, etc. The filler can be either one component or two components. Two components filler usually has a resin and a curing agent. They are mixed together either before injection or during the injection. The physical means often involve change in temperature, light, electricity, pH, ionic strength, concentration, etc. A typical material that can be triggered by the temperature change is Pluronic. After the filler is hardened, the liner can provide additional strength to the aneurysm wall and maintain the shape of the liner to ensure close contact with the inner surface of aneurysm. Alternatively, the filler in the inflatable chambers facing the aneurysm wall remains soft to enhance the liner's ability to cushion the aneurysm wall. On the other hand, the filler in the inflatable chambers facing the flow conduit is hardened and provides additional support to the flow conduit. Exemplary non-limiting examples include silicone, polydimethylsiloxane, polysiloxane rubber, hydrogel, polyurethane, cyanoacrylate, methacrylate, acrylate, polymethylmethacrylate, polybutylmethacrylate, polyhydroxy ethyl acrylate, polyhydroxy ethyl methacrylate, poly(hydroxy ethyl acrylate), poly(hydroxy ethyl methacrylate), polymethacrylic acid, polyacrylic acid, polyesters, polybutester, polyacrylamide, polyacrylamide copolymer, sodium acrylate and vinyl alcohol copolymer, polyvinyl alcohol, polyacetals, polyvinyl acetate, acrylic acid ester copolymer, polyvinyl pyrrolidone, polyacrylonitrile, polyarylethernitriles, Hypan, poly(2-hydroxyethyl methacrylate)(polyHEMA), Carbomer copolymer and homopolymer, alkoxylated surfactants, alkylphenol ethoxylates, ethoxylated fatty acids, alcohol ethoxylates, alcohol alkoxylates, polyethylene oxide, poly(propylene oxide), polyethylene oxide, poly(ethylene glycol), poly(propylene glycol), poly(vinylcarboxylic acid), collagen, polyvinyl pyridine, polylysine, polyarginine, poly aspartic acid, poly glutamic acid, polytetramethylene oxide, methoxylated pectin gels, cellulose acetate phthalate, gelatin, alginate, calcium alginate, Carbopol, Poloxamer, Pluronic, Tetronics, PEO-PPO-PEO triblocks copolymer, Tetrafunctional block copolymer of PEO-PPO condensed with ethylenadiamine, Poly(acrylic acid) grafted (PEO-PPO-PEO-PAA) copolymers, graft copolymers of Pluronic and poly(acrylic acid), ethyl(hydroxyethyl) cellulose (EHEC) formulated with ionic surfactants, alkylcellulose, hydroxyalkylcellulose, PEG-PLA-PEG block polymers, Poly(N-isopropylacrylamide)(PNIPAAm), tetrafunctional block copolymer of PEO-PPO-ethylenadiamine, copolymer of PNIPAAm and acrylic acid (AAc), P(NIPAAm-co-AAc) and the oligomer and monomer of above.

In another embodiment according to the present invention, the filler includes a bioactive or a pharmaceutical agent. The bioactive or pharmaceutical agent can be mixed with filler before injection in the liner. After the inflation, the agent diffuses into the aneurysm wall and treats the disease in the vessel. Because the multiple walls liner of this invention is in close contact with the aneurysm wall, the agent can reach the aneurysm wall without being diluted by the blood. Dilution decreases the efficacy of the agent when it is delivered orally or by injection. Many bioactive or pharmaceutical agents can be mixed with filler to treat aneurysm in this invention. Agents that inhibit matrix metalloproteinases, inflammation or other pathological processes involved in aneurysm progression, can be incorporated into the filler to enhance wound healing, stabilize and possibly reverse the pathology of aneurysm. Agents that induce positive effects at the aneurysm site, such as growth factor, can also be delivered by the filler and the methods described herein. Exemplary non-limiting examples include platelet-derived growth factor (PDGF), platelet-derived epidermal growth factor (PDEGF), fibroblast growth factor (FGF), transforming growth factor-beta (TGF-β), platelet-derived angiogenesis growth factor (PDAF), transforming growth factor-beta (TGF-β), basic fibroblast growth factor (bFGF), vascular growth factor, vascular endothelial growth factor, and placental growth factor. These agents have been implicated in wound healing by increasing collagen secretion, vascular growth and fibroblast proliferation. Other exemplary non-limiting examples include Doxycycline, Tetracycline, peptides, proteins, hormones, DNA or RNA fragments, genes, cells, cell growth promoting compositions, and autologous platelet gel (APG). Alternatively, the bioactive or pharmaceutical agent can be coated on the outer surface of the liner. The agent or cell growth promoting factor on the outer surface of liner can activate cell growth and proliferation. Those cells adhere to the liner and anchor the liner securely to the vessel lumen and thus preventing migration. Moreover, tissue in-growth on the liner can also provide a seal around the junction of collateral arteries in the aneurysm and prevent endoleak.

In another embodiment of the present invention, the outer wall of the liner is treated to increase its surface area. The increased surface area can increase the contact between the vessel and the liner. Due to the intimate contact with the outer surface of the liner, smooth muscle cells and fibroblasts, etc. in the vessel will be stimulated to proliferate. As these cells proliferate they will grow onto the outer wall of the liner so that the outer wall becomes physically attached to the vessel lumen. The attached cells or tissue on the liner wall can enhance the bonding and seal between the vessel wall and the liner. Increased surface area on the outer wall can further enhance the contact between the vessel and the liner and stimulate more cells proliferate and bonding. In addition, the increase surface area also promotes the formation of thrombosis. The thrombosis can fill gaps between the outer wall of the liner and the surface of the aneurysm wall further preventing endoleak. Typical techniques to increase surface area are sanding, etching, depositing, coating, bonding with fibers or thin foam. Fibers such as PET fibrils are biocompatible with high surface area. They are well-known to the people skilled in the art.

There are several benefits for this present invention to treat aneurysm. First, the liner can strengthen the aneurysm wall and prevent the rupture of aneurysm by reducing the hemodynamic pressure on the aneurysm wall. Second, the collapsed liner is flexible so that it can be easily loaded in a catheter and access the aneurysm site via iliac artery and then deployed in the aneurysm with minimum invasivity. Third, the flexibility of the liner and the radial force provided by the hemodynamic force allow the liner to conform to the inner surface of the aneurysm wall without gap between them. After hardening of filler, the liner will be “locked” in the aneurysm without endoleak or migration. Fourth, less filler is required to cover the inner surface of aneurysm wall than filling the whole aneurysm. The resulting liner is more flexible than the filler structure that fills the whole aneurysm. This flexible liner is more compatible with the vessel and adjacent organs. Fifth, there is no excess amount of stress on the aneurysm wall during the inflation of the liner. In order to prevent endoleak and migration, it is essential to have close contact between the outer wall of the liner and the surface of the aneurysm wall. As what was disclosed in the prior arts, the whole aneurysm (other than the tubular flow conduit within the aneurysm) needs to be filled to achieve that. Insufficient filler will result in gaps between the liner and the surface of the aneurysm wall. On the other hand, too much filler will place excess circumferential stress on the weak aneurysm wall. However, because the gap and the aneurysm wall have no contrast agent in them and can't be visualized under Fluoroscope, physician cannot determine if the gap has been filled (or not being filled) by the fill structure during the inflation of the fill structure. This uncertainty can place the patient in great risk. Additionally, as described in prior arts, the aneurysm is usually sealed by a stent graft or a lumen shaping balloon before the fill structure is inflated. Existing blood in the aneurysm (with the added filler) can also cause high stress on the aneurysm wall during the inflation of fill structure if the collateral arteries in the aneurysm are occluded. In the present invention, the close contact between the aneurysm wall and the outer wall of the liner is a result of flexible walls and the radial expanding force provided by the hemodynamic force. It is not necessary to fill the whole aneurysm in order to close the gap between the aneurysm wall and the liner. As a result, the systems and methods provided by this present invention are safer than what were disclosed in the prior arts. Sixth, the present invention can enhance the adhesion of the liner to the aneurysm wall to further reduce the risk of liner migration and endoleak. Seventh, this invention enables the use of bioactive or pharmaceutical agents in the filler to treat aneurysm

1. A system to protect the wall of an aneurysm in a vessel wherein the system comprises:
a liner comprising one or more inflatable chambers for the introduction of an inflation medium, said one or more inflatable chambers comprising one or more connectors, wherein said liner is configured to conform to the interior surface of the aneurysm following introduction of said liner into the vessel, and wherein said one or more connectors constrains expansion of said one or more chambers upon introduction of said inflation medium.

2. The system as set forth in claim 1 further comprising means for anchoring said liner to the interior of the vessel.

3. The system as set forth in claim 2, wherein said means for anchoring said liner comprises one or more expandable elements coupled to said liner.

4. The system as set forth in claim 3, wherein said one or more expandable elements comprises a stent.

5. The system as set forth in claim 1, wherein one or more of said inflatable chambers comprises one or more opposing interior walls and said one or more connectors is affixed to opposing interior walls.

6. The system as set forth in claim 5, wherein said one or more connectors comprises a strip, a string, or a bond.

7. The system as set forth in claim 1, wherein said one or more inflatable chambers comprises an inflatable patch or an inflatable channel.

8. The system as set forth in claim 1, wherein said one or more inflatable chambers is disposed helically on the exterior of said liner.

9. The system as set forth in claim 1, wherein said one or more inflatable chambers is disposed circumferentially on the exterior of said liner.

10. The system as set forth in claim 1, wherein the said liner comprises flexible and substantially inelastic biocompatible material.

11. The system as set forth in claim 1, wherein one or more inflatable chambers is in fluid communication with one or more adjacent inflatable chambers.

12. The system as set forth in claim 1 further comprising a one way valve in fluid communication with one or more inflatable chambers.

13. The system as set forth in claim 1, wherein said liner comprises an inner wall defining a main flow conduit of the vessel proximate the aneurysm following introduction of the liner into the vessel, said conduit comprising an inlet and one or more outlets.

14. The system as set forth in claim 13, wherein said main flow conduit is defined by the inner surface of the aneurysm, said connectors and the amount inflation medium in said liner.

15. The system as set forth in claim 1, wherein the inflation medium comprises a fluid comprising a polymer, an oligomer or a monomer.

16. The system as set forth in claim 1, wherein the inflation medium comprises a fluid selected from the group consisting of silicone, hydrogel, saline, water, blood, polyvinyl alcohol, cyanoacrylate, methacrylate, acrylate, polyacrylic acid polymer, polyacrylamide, polyvinyl pyrrolidone, polyacrylonitrile, Hypan, poly(2-hydroxyethyl methacrylate), polyethylene oxide, poly(propylene oxide), poly(ethylene glycol), poly(propylene glycol), Poloxamer, Pluronic, and Tetronics.

17. The system as set forth in claim 1 wherein said inflation medium comprises a fluid, and wherein the fluid is curable by either chemical or physical means after injection into the liner.

18. The system as set forth in claim 1 wherein said inflation medium comprises a fluid, and said fluid comprises a bioactive or a pharmaceutical active component.

19. The system as set forth in claim 1, wherein the liner comprises an outer surface comprising a bioactive or a pharmaceutical active component.

20. The system as set forth in claim 1, wherein the liner comprises an outer surface and surface area, wherein said outer surface is treated with fibers, fibril, foam, or roughening to increase the surface area.

21. The system as set forth in claim 1 further comprising means to introduce a hemodynamic force in said liner whereby said liner expands and conforms to the interior surface of the aneurysm.

22. A system to protect the wall of an aneurysm in a vessel wherein the system comprises:
an inflatable multiple walls liner having an inner wall and an outer wall, wherein said inner wall and outer wall being connected by one or more connectors to form one or more inflatable chambers to be filled by an inflation medium, wherein said liner is configured to conform to the interior surface of the aneurysm by a hemodynamic force in the vessel, and wherein said one or more connectors constrains expansion of said one or more chambers upon introduction of said inflation medium.

23. The system as set forth in claim 22 further comprising means for anchoring said liner to the interior of the vessel.

24. The system as set forth in claim 23, wherein said means for anchoring said liner comprises one or more expandable elements coupled to said liner.

25. A method of treatment of an aneurysm comprising:
providing an inflatable liner comprising one or more inflatable chambers; and
anchoring a portion of the inflatable liner in the vessel adjacent the aneurysm with a first expandable element and
introducing hemodynamic force in the inflatable liner whereby said liner expands and conforms to the interior surface of the aneurysm.
introducing inflation medium into the one or more inflatable chambers whereby said liner expands and protects the vessel.

26. A method of treatment of an aneurysm of a subject vessel wherein the vessel comprises an interior, an interior surface and a hemodynamic force therethrough, the method comprising:
providing an inflation medium, one or more expandable elements and an inflatable liner comprising one or more inflatable chambers, one or more flow conduit outlets;
introducing said inflatable liner into the vessel;
anchoring the inflatable liner within the vessel proximate the aneurysm via deployment of one or more expandable elements;
permitting the hemodynamic force to expand said inflatable liner to substantially conform to the interior surface of the vessel;
securing patency of one or more of said flow conduit outlets via deployment of one or more expandable elements;
introducing said inflation medium into the one or more inflatable chambers; and removing one or more expandable elements.

27. The method as set forth in claim 26 further comprising the steps of:
providing one or more stents;
introducing one or more stents into the inflatable liner; and
deploying one or more stents within the inflatable liner.

28. The method as set forth in claim 26, wherein said step of anchoring said inflatable liner via deployment of said expandable element permits fluid perfusion therethrough and substantially prevents fluid flow between said inflatable liner and the interior surface of said vessel following deployment of said expandable element.

29. The method as set forth in claim 28, wherein said expandable element is a balloon, a balloon comprising an annular shape or a stent.

30. The method as set forth in claim 26, wherein said one or more expandable elements in said step of securing patency of one or more of said flow conduit outlets comprise a balloon, a balloon comprising an annular shape, or a stent.

31. The method as set forth in claim 26, wherein said inflatable liner further comprising one or more stents.

32. The method as set forth in claim 26 further comprising the step of;
allowing the perfusion of fluid between said inflatable liner and said one or more expandable elements via reducing the size of said one or more expandable elements.

33. A method of treatment of an aneurysm of a subject vessel wherein the vessel comprises an interior, an interior surface and a hemodynamic force therethrough, the method comprising:
providing an inflation medium, one or more expandable elements and an inflatable liner comprising one or more inflatable chambers, one or more flow conduit outlets and one or more stents;
introducing said inflatable liner into the vessel;
anchoring the inflatable liner within the vessel proximate the aneurysm via deployment of one or more stents;
permitting the bemodynamic force to expand said inflatable liner to substantially conform to the interior surface of the vessel;
securing patency of one or more of said flow conduit outlets via deployment of one or more expandable elements;
introducing said inflation medium into the one or more inflatable chambers; and removing one or more expandable elements.

34. The method as set forth in claim 33 further comprising the steps of:
providing one or more stents;
introducing one or more stents into the inflatable liner; and
deploying one or more stents within the inflatable liner.

35. The method as set forth in claim 33, wherein said step of anchoring said inflatable liner via deployment of said one or more stents permits fluid perfusion therethrough and substantially prevents fluid flow between said inflatable liner and the interior surface of said vessel following deployment of said one or more stents.

36. The method as set forth in claim 33, wherein said one or more expandable elements in said step of securing patency of one or more of said flow conduit outlets comprise a balloon, a balloon comprising an annular shape, or a stent.

37. The method as set forth in claim 33 further comprising the steps of:
deploying one or more expandable elements within the inflatable liner proximate the aneurysm;
allowing the perfusion of fluid between said inflatable liner and said one or more expandable elements via reducing the size of said one or more expandable elements.
